(19) United States
(12) Patent Application Publication
(10) Pub. No.: US 2025/0129032 A1
(43) Pub. Date: Apr. 24, 2025

(54) PROTEIN TYROSINE PHOSPHATASE INHIBITORS AND USES THEREOF

(71) Applicant: Nerio Therapeutics, Inc., La Jolla, CA (US)

(72) Inventors: Jason ROLAND, San Diego, CA (US); Sergio G. DURON, San Diego, CA (US)

(21) Appl. No.: 18/834,765

(22) PCT Filed: Feb. 1, 2023
(86) PCT No.: PCT/US2023/061714

§ 371 Date: Jul. 31, 2024

Related U.S. Application Data
(60) Provisional application No. 63/305,789, filed on Feb. 2, 2022.

(51) Int. Cl. C07D 285/10; A61K 31/433; A61K 31/55; A61K 39/395; C07D 417/04; C07D 417/10

(52) U.S. Cl. CPC C07D 285/10; A61K 31/433; A61K 31/55; A61K 39/3955; C07D 417/04; C07D 417/10

(57) ABSTRACT

Provided herein are compounds, compositions, and methods useful for inhibiting protein tyrosine phosphatase, e.g., protein tyrosine phosphatase non-receptor type 2 (PTPN2) and/or protein tyrosine phosphatase non-receptor type 1 (PTPN1), and for treating related diseases, disorders, and conditions favorably responsive to PTPN1 or PTPN2 inhibitor treatment, e.g., a cancer or a metabolic disease.

### CROSS-REFERENCE

This application claims the benefit of U.S. Provisional Application Ser. No. 63/305,789 filed Feb. 2, 2022 which is hereby incorporated by reference in its entirety.

### BACKGROUND OF THE INVENTION

Cancer immunotherapy regimens targeting immune evasion mechanisms including checkpoint blockade (e.g., PD-1/PD-L1 and CTLA-4 blocking antibodies) have been shown to be effective in treating in a variety of cancers, dramatically improving outcomes in some populations refractory to conventional therapies. However, incomplete clinical responses and the development of intrinsic or acquired resistance continue to limit the patient populations who could benefit from checkpoint blockade.

Protein tyrosine phosphatase non-receptor type 2 (PTPN2), also known as T cell protein tyrosme phosphatase (TC-PTP), is an intercellular member of the class 1 subfamily of phospho-tyrosine specific phosphatases that control multiple cellular regulatory processes by removing phosphate groups from tyrosine substrates PTPN2 is ubiquitously expressed, but expression is highest in hematopoietic and placental cells. In humans, PTPN2 expression is controlled post-transcriptionally by the existence of two splice variants: a 45 kDa form that contains a nuclear localization signal at the C-terminus upstream of the splice junction, and a 48 kDa canonical form which has a C-terminal ER retention motif. The 45 kDa isoform can passively transfuse into the cytosol under certain cellular stress conditions. Both isoforms share an N-terminal phospho-tyrosine phosphatase catalytic domain. PTPN2 negatively regulates signaling of non-receptor tyrosine kinases (e.g., JAK1. JAK3), receptor tyrosine kinases (e.g., INSR, EGFR, CSFIR, PDGFR), transcription factors (e.g., STAT1, STAT3, STAT5a/b), and Src family kinases (e.g., Fyn, Lck). As a critical negative regulator of the JAK-STAT pathway, PTPN2 functions to directly regulate signaling through cytokine receptors, including IFNγ. The PTPN2 catalytic domain shares 74% sequence homology with PTPN1 (also called PTP1B), and shares similar enzymatic kinetics. Data from a loss of function in vivo genetic screen using CRISPR/Cas9 genome editing in a mouse B16F10 transplantable tumor model show that deletion of Ptpn2 gene in tumor cells improved response to the immunotherapy regimen of a GM-CSF secreting vaccine (GVAX) plus PD-1 checkpoint blockade. Loss of PTPN2 sensitized tumors to immunotherapy by enhancing IFNγ-mediated effects on antigen presentation and growth suppression. The same screen also revealed that genes known to be involved in immune evasion, including PD-L1 and CD47, were also depleted under immunotherapy selective pressure, while genes involved in the IFNγ signaling pathway, including IFNGR, JAK1, and STAT1, were enriched. These observations point to a putative role for therapeutic strategies that enhance IFNγ sensing and signaling in enhancing the efficacy of cancer immunotherapy regimens.

Protein tyrosine phosphatase non-receptor type 1 (PTPN1), also known as protein tyrosme phosphatase-1B (PTP1B), has been shown to play a key role in insulin and leptin signaling and is a primary mechanism for down-regulating both the insulin and leptin receptor signaling pathways Animals deficient in PTP1B have improved glucose regulation and lipid profiles and are resistant to weight gain when treated with a high fat diet. Thus. PTP1B inhibitors are expected to be useful for the treatment of type 2 diabetes, obesity, and metabolic syndrome.

### SUMMARY OF THE INVENTION

Disclosed herein is a compound of Formula (I), or a pharmaceutically acceptable salt, solvate, or stereoisomer thereof:

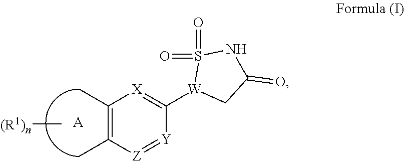

wherein:
Ring A is a 7- to 15-membered cycloalkyl or a 7- to 15-membered heterocycloalkyl comprising 1 to 4 heteroatoms selected from O, S, and N;
each R¹ is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{b}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl; wherein each alkyl, cycloalkyl, heterocycloalkyl, aryl, and heteroaryl is independently and optionally substituted with one or more R^{1a};
or two R¹ on the same atom are taken together to form an oxo;
each R^{1a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl; wherein each alkyl, cycloalkyl, heterocycloalkyl, aryl, and heteroaryl is independently and optionally substituted with one or more R;
or two R^{1a} on the same atom are taken together to form an oxo;
n is 0-6;
X is CR^{X} or N;
R^{X} is hydrogen, deuterium, halogen, —CN, —NO₂, —OH, —OR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl;
Y is CR^{Y} or N;
R^{Y} is hydrogen, deuterium, halogen, —CN, —NO₂, —OH, —OR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl;
Z is CR^{Z} or N;
R^{Z} is hydrogen, deuterium, halogen, —CN, —NO₂, —OH, —OR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl;
W is CR^{W} or N;
R^{W} is hydrogen, deuterium, halogen, —CN, —NO₂, —OH, —OR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl;
each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently and optionally substituted with one or more R;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently and optionally substituted with one or more R;
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently and optionally substituted with one or more R;
or R^{c} and R^{d} are taken together with the atom to which they are attached to form a heterocycloalkyl optionally substituted with one or more R; and
each R is independently deuterium, halogen, —CN, —OH, —OC₁-C₆alkyl, —S(═O)C₁-C₆alkyl, —S(═O)₂C₁-C₆alkyl, —S(═O)₂NH₂, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl; or two R on the same atom are taken together to form an oxo.

Also disclosed herein is a pharmaceutical composition comprising a compound disclosed herein, or a pharmaceutically acceptable salt, solvate, or stereoisomer thereof, and a pharmaceutically acceptable excipient.

Also disclosed herein is a method of treating cancer in a subject in need thereof, the method comprising administering to the subject an effective amount of a compound disclosed herein, or a pharmaceutically acceptable salt, solvate, or stereoisomer thereof.

Also disclosed herein is a method of treating cancer in a subject in need thereof, the method comprising administering to the subject a pharmaceutical composition disclosed herein.

In some embodiments, the method further comprises administering an additional therapeutic agent. In some embodiments, the additional therapeutic agent is an immunotherapeutic agent. In some embodiments, the immunotherapeutic agent is an anti-PD-1 antibody, an anti-PD-L1 antibody, or an anti-CTLA-4 antibody.

Also disclosed herein is a method of treating type-2 diabetes in a subject in need thereof, the method comprising administering to the subject an effective amount of a compound disclosed herein, or a pharmaceutically acceptable salt, solvate, or stereoisomer thereof.

Also disclosed herein is a method of treating type-2 diabetes in a subject in need thereof, the method comprising administering to the subject a pharmaceutical composition disclosed herein.

Also disclosed herein is a method of treating and/or controlling obesity in a subject in need thereof, the method comprising administering to the subject an effective amount of a compound disclosed herein, or a pharmaceutically acceptable salt, solvate, or stereoisomer thereof.

Also disclosed herein is a method of treating and/or controlling obesity in a subject in need thereof, the method comprising administering to the subject a pharmaceutical composition disclosed herein.

Also disclosed herein is a method of treating a metabolic disease in a subject in need thereof, the method comprising administering to the subject an effective amount of a compound disclosed herein, or a pharmaceutically acceptable salt, solvate, or stereoisomer thereof.

Also disclosed herein is a method of treating a metabolic disease in a subject in need thereof, the method comprising administering to the subject a pharmaceutical composition disclosed herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

### Definitions

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “some embodiments” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The terms below, as used herein, have the following meanings, unless indicated otherwise:

“oxo” refers to ═O.

“Carboxyl” refers to —COOH.

“Cyano” refers to —CN.

“Alkyl” refers to a straight-chain, or branched-chain saturated hydrocarbon monoradical having from one to about ten carbon atoms, more preferably one to six carbon atoms. Examples include, but are not limited to methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl and the like. Whenever it appears herein, a numerical range such as “C₁-C₆ alkyl” or “C₁₋₆alkyl”, means that the alkyl group may consist of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁₋₁₀alkyl. In some embodiments, the alkyl is a C₁₋₆alkyl. In some embodiments, the alkyl is a C₁₋₅alkyl. In some embodiments, the alkyl is a C₁₋₄alkyl. In some embodiments, the alkyl is a C₁₋₃alkyl. Unless stated otherwise specifically in the specification, an alkyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkyl is optionally substituted with oxo, halogen, —CN, —COOH, —COOMe, —OH, —OMe, —NH₂, or —NO₂. In some embodiments, the alkyl is optionally substituted with halogen, —CN, —OH, or —OMe. In some embodiments, the alkyl is optionally substituted with halogen.

“Alkenyl” refers to a straight-chain, or branched-chain hydrocarbon monoradical having one or more carbon-carbon double-bonds and having from two to about ten carbon atoms, more preferably two to about six carbon atoms. The group may be in either the cis or trans conformation about the double bond(s), and should be understood to include both isomers. Examples include, but are not limited to ethenyl (—CH═CH₂), 1-propenyl (—CH₂CH═CH₂), isopropenyl [—C(CH₃)═CH₂], butenyl, 1,3-butadienyl and the like. Whenever it appears herein, a numerical range such as “C₂-C₆ alkenyl” or “C₂₋₆alkenyl”, means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. Unless stated otherwise specifically in the specification, an alkenyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkenyl is optionally substituted with oxo, halogen, —CN, —COOH, —COOMe, —OH, —OMe, —NH₂, or —NO₂. In some embodiments, the alkenyl is optionally substituted with halogen, —CN, —OH, or —OMe. In some embodiments, the alkenyl is optionally substituted with halogen.

“Alkynyl” refers to a straight-chain or branched-chain hydrocarbon monoradical having one or more carbon-carbon triple-bonds and having from two to about ten carbon atoms, more preferably from two to about six carbon atoms. Examples include, but are not limited to ethynyl, 2-propynyl, 2-butynyl, 1,3-butadiynyl and the like. Whenever it appears herein, a numerical range such as “C₂-C₆ alkynyl” or “C₂₋₆alkynyl”, means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated. Unless stated otherwise specifically in the specification, an alkynyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkynyl is optionally substituted with oxo, halogen, —CN, —COOH, COOMe, —OH, —OMe, —NH₂, or —NO₂. In some embodiments, the alkynyl is optionally substituted with halogen, —CN, —OH, or —OMe. In some embodiments, the alkynyl is optionally substituted with halogen.

“Alkylene” refers to a straight or branched divalent hydrocarbon chain. Unless stated otherwise specifically in the specification, an alkylene group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkylene is optionally substituted with oxo, halogen, —CN, —COOH, COOMe, —OH, —OMe, —NH₂, or —NO₂. In some embodiments, the alkylene is optionally substituted with halogen, —CN, —OH, or —OMe. In some embodiments, the alkylene is optionally substituted with halogen.

“Alkoxy” refers to a radical of the formula —ORₐ where Rₐ is an alkyl radical as defined. Unless stated otherwise specifically in the specification, an alkoxy group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkoxy is optionally substituted with halogen, —CN, —COOH, COOMe, —OH, —OMe, —NH₂, or —NO₂. In some embodiments, the alkoxy is optionally substituted with halogen, —CN, —OH, or —OMe. In some embodiments, the alkoxy is optionally substituted with halogen.

“Aryl” refers to a radical derived from a hydrocarbon ring system comprising 6 to 30 carbon atoms and at least one aromatic ring. The aryl radical may be a monocyclic, bicyclic, tricyclic, or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl (phenyl). Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the aryl is optionally substituted with halogen, methyl, ethyl, —CN, —COOH, COOMe, —CF₃, —OH, —OMe, —NH₂, or —NO₂. In some embodiments, the aryl is optionally substituted with halogen, methyl, ethyl, —CN, —CF₃, —OH, or —OMe. In some embodiments, the aryl is optionally substituted with halogen.

“Cycloalkyl” refers to a partially or fully saturated, monocyclic, or polycyclic carbocyclic ring, which may include fused (when fused with an aryl or a heteroaryl ring, the cycloalkyl is bonded through a non-aromatic ring atom) or bridged ring systems. In some embodiments, the cycloalkyl is fully saturated. Representative cycloalkyls include, but are not limited to, cycloalkyls having from three to fifteen carbon atoms (C₃-C₁₅ cycloalkyl or C₃-C₁₅ cycloalkenyl), from three to ten carbon atoms (C₃-C₁₀cycloalkyl or C₃-C₁₀ cycloalkenyl), from three to eight carbon atoms (C₃-C₈ cycloalkyl or C₃-C₈ cycloalkenyl), from three to six carbon atoms (C₃-C₆ cycloalkyl or C₃-C₆ cycloalkenyl), from three to five carbon atoms (C₃-C₅ cycloalkyl or C₃-C₅ cycloalkenyl), or three to four carbon atoms (C₃-C₄ cycloalkyl or C₃-C₄ cycloalkenyl). In some embodiments, the cycloalkyl is a 3- to 10-membered cycloalkyl or a 3- to 10-membered cycloalkenyl. In some embodiments, the cycloalkyl is a 3- to 6-membered cycloalkyl or a 3- to 6-membered cycloalkenyl. In some embodiments, the cycloalkyl is a 5-to 6-membered cycloalkyl or a 5- to 6-membered cycloalkenyl. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, a cycloalkyl is optionally substituted with oxo, halogen, methyl, ethyl, —CN, —COOH, COOMe, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Halo” or “halogen” refers to bromo, chloro, fluoro or iodo. In some embodiments, halogen is fluoro or chloro. In some embodiments, halogen is fluoro.

“Haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxypentyl. In some embodiments, the hydroxyalkyl is hydroxymethyl.

“Aminoalkyl” refers to an alkyl radical, as defined above, that is substituted by one or more amines. In some embodiments, the alkyl is substituted with one amine. In some embodiments, the alkyl is substituted with one, two, or three amines. Aminoalkyl include, for example, aminomethyl, aminoethyl, aminopropyl, aminobutyl, or aminopentyl. In some embodiments, the aminoalkyl is aminomethyl.

“Deuteroalkyl” refers to an alkyl radical, as defined above, that is substituted by one or more deuteriums. In some embodiments, the alkyl is substituted with one deuterium. In some embodiments, the alkyl is substituted with one, two, or three deuteriums. In some embodiments, the alkyl is substituted with one, two, three, four, five, or six deuteriums. Deuteroalkyl include, for example, CD₃, CH₂D, CHD₂, CH₂CD₃, CD₂CD₃, CHDCD₃, CH₂CH₂D, or CH₂CHD₂. In some embodiments, the deuteroalkyl is CD₃.

“Heteroalkyl” refers to an alkyl group in which one or more skeletal atoms of the alkyl are selected from an atom other than carbon, e.g., oxygen, nitrogen (e.g., —NH—, —N(alkyl)-), sulfur, phosphorus, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, phosphorus, or combinations thereof wherein the heteroalkyl is attached to the rest of the molecule at a carbon atom of the heteroalkyl. Examples of such heteroalkyl are, for example, —CH₂OCH₃, —CH₂CH₂OCH₃, —CH₂CH₂OCH₂CH₂OCH₃, —CH(CH₃)OCH₃, —CH₂NHCH₃, —CH₂N(CH₃)₂, —CH₂CH₂NHCH₃, or —CH₂CH₂N(CH₃)₂. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

“Heterocycloalkyl” refers to a 3- to 24-membered partially or fully saturated ring radical comprising 2 to 23 carbon atoms and from one to 8 heteroatoms selected from the group consisting of nitrogen, oxygen, phosphorous and sulfur. In some embodiments, the heterocycloalkyl is fully saturated. In some embodiments, the heterocycloalkyl comprises one to three heteroatoms selected from the group consisting of nitrogen, oxygen, and sulfur. In some embodiments, the heterocycloalkyl comprises one to three heteroatoms selected from the group consisting of nitrogen and oxygen. In some embodiments, the heterocycloalkyl comprises one to three nitrogens. In some embodiments, the heterocycloalkyl comprises one or two nitrogens. In some embodiments, the heterocycloalkyl comprises one nitrogen. In some embodiments, the heterocycloalkyl comprises one nitrogen and one oxygen. Unless stated otherwise specifically in the specification, the heterocycloalkyl radical may be a monocyclic, bicyclic, tricyclic, or tetracyclic ring system, which may include fused (when fused with an aryl or a heteroaryl ring, the heterocycloalkyl is bonded through a non-aromatic ring atom) or bridged ring systems; and the nitrogen, carbon, or sulfur atoms in the heterocycloalkyl radical may be optionally oxidized; the nitrogen atom may be optionally quaternized. Representative heterocycloalkyls include, but are not limited to, heterocycloalkyls having from two to fifteen carbon atoms (C₂-C₁₅ heterocycloalkyl or C₂-C₁₅ heterocycloalkenyl), from two to ten carbon atoms (C₂-C₁₀ heterocycloalkyl or C₂-C₁₀ heterocycloalkenyl), from two to eight carbon atoms (C₂-C₈ heterocycloalkyl or C₂-C₈ heterocycloalkenyl), from two to seven carbon atoms (C₂-C₇ heterocycloalkyl or C₂-C₇ heterocycloalkenyl), from two to six carbon atoms (C₂-C₆ heterocycloalkyl or C₂-C₆ heterocycloalkenyl), from two to five carbon atoms (C₂-C₅ heterocycloalkyl or C₂-C₅ heterocycloalkenyl), or two to four carbon atoms (C₂-C₄ heterocycloalkyl or C₂-C₄ heterocycloalkenyl). Examples of such heterocycloalkyl radicals include, but are not limited to, aziridinyl, azetidinyl, oxetanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to the monosaccharides, the disaccharides, and the oligosaccharides. Unless otherwise noted, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). In some embodiments, the heterocycloalkyl is a 3- to 8-membered heterocycloalkyl. In some embodiments, the heterocycloalkyl is a 3- to 7-membered heterocycloalkyl. In some embodiments, the heterocycloalkyl is a 3- to 6-membered heterocycloalkyl. In some embodiments, the heterocycloalkyl is a 4- to 6-membered heterocycloalkyl. In some embodiments, the heterocycloalkyl is a 5- to 6-membered heterocycloalkyl. In some embodiments, the heterocycloalkyl is a 3- to 8-membered heterocycloalkenyl. In some embodiments, the heterocycloalkyl is a 3- to 7-membered heterocycloalkenyl. In some embodiments, the heterocycloalkyl is a 3- to 6-membered heterocycloalkenyl. In some embodiments, the heterocycloalkyl is a 4- to 6-membered heterocycloalkenyl. In some embodiments, the heterocycloalkyl is a 5- to 6-membered heterocycloalkenyl. Unless stated otherwise specifically in the specification, a heterocycloalkyl may be optionally substituted as described below, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heterocycloalkyl is optionally substituted with oxo, halogen, methyl, ethyl, —CN, —COOH, COOMe, —CF₃, —OH, —OMe, —NH₂, or —NO₂. In some embodiments, the heterocycloalkyl is optionally substituted with halogen, methyl, ethyl, —CN, —CF₃, —OH, or —OMe. In some embodiments, the heterocycloalkyl is optionally substituted with halogen.

“Heteroaryl” refers to a 5- to 14-membered ring system radical comprising one to thirteen carbon atoms, one to six heteroatoms selected from the group consisting of nitrogen, oxygen, phosphorous, and sulfur, and at least one aromatic ring. In some embodiments, the heteroaryl comprises one to three heteroatoms selected from the group consisting of nitrogen, oxygen, and sulfur. In some embodiments, the heteroaryl comprises one to three heteroatoms selected from the group consisting of nitrogen and oxygen. In some embodiments, the heteroaryl comprises one to three nitrogens. In some embodiments, the heteroaryl comprises one or two nitrogens. In some embodiments, the heteroaryl comprises one nitrogen. The heteroaryl radical may be a monocyclic, bicyclic, tricyclic, or tetracyclic ring system, which may include fused (when fused with a cycloalkyl or heterocycloalkyl ring, the heteroaryl is bonded through an aromatic ring atom) or bridged ring systems; and the nitrogen, carbon, or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. In some embodiments, the heteroaryl is a 6-membered heteroaryl. In some embodiments, the heteroaryl is a 5-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heteroaryl is optionally substituted with halogen, methyl, ethyl, —CN, —COOH, COOMe, —CF₃, —OH, —OMe, —NH₂, or —NO₂. In some embodiments, the heteroaryl is optionally substituted with halogen, methyl, ethyl, —CN, —CF₃, —OH, or —OMe. In some embodiments, the heteroaryl is optionally substituted with halogen.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group may be un-substituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc.). It will be understood by those skilled in the art with respect to any group containing one or more substituents that such groups are not intended to introduce any substitution or substitution patterns (e.g., substituted alkyl includes optionally substituted cycloalkyl groups, which in turn are defined as including optionally substituted alkyl groups, potentially ad infinitum) that are sterically impractical and/or synthetically non-feasible. Thus, any substituents described should generally be understood as having a maximum molecular weight of about 1,000 daltons, and more typically, up to about 500 daltons.

The term “one or more” when referring to an optional substituent means that the subject group is optionally substituted with one, two, three, or four substituents. In some embodiments, the subject group is optionally substituted with one, two, or three substituents. In some embodiments, the subject group is optionally substituted with one or two substituents. In some embodiments, the subject group is optionally substituted with one substituent. In some embodiments, the subject group is optionally substituted with two substituents.

An “effective amount” or“therapeutically effective amount” refers to an amount of a compound administered to a mammalian subject, either as a single dose or as part of a series of doses, which is effective to produce a desired therapeutic effect.

“Treatment” of an individual (e.g. a mammal, such as a human) or a cell is any type of intervention used in an attempt to alter the natural course of the individual or cell. In some embodiments, treatment includes administration of a pharmaceutical composition, subsequent to the initiation of a pathologic event or contact with an etiologic agent and includes stabilization of the condition (e.g., condition does not worsen) or alleviation of the condition.

“Synergy” or “synergize” refers to an effect of a combination that is greater than additive of the effects of each component alone at the same doses.

As used herein, the term “PTPN2-mediated” disorder or disease or alternatively “disease or disorder associated with PTPN2” means any disease or other deleterious condition in which PTPN2 or a mutant thereof is known to play a role. Accordingly, in some embodiments, the methods relate to treating or lessening the severity of one or more diseases in which PTPN2, or a mutant thereof, is known to play a role.

As used herein, the term “PTPN1-mediated” disorder or disease or alternatively “disease or disorder associated with PTPN1” means any disease or other deleterious condition in which PTPN1 or a mutant thereof is known to play a role. Accordingly, in some embodiments, the methods relate to treating or lessening the severity of one or more diseases in which PTPN1, or a mutant thereof, is known to play a role.

### Compounds

Described herein are compounds of Formula (I), or a pharmaceutically acceptable salt, solvate, or stereoisomer thereof, that are dual PTPN1/PTPN2 inhibitors.

Disclosed herein is a compound of Formula (I), or a pharmaceutically acceptable salt, solvate, or stereoisomer thereof:

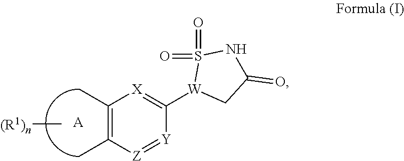

wherein:
Ring A is a 7- to 15-membered cycloalkyl or a 7- to 15-membered heterocycloalkyl comprising 1 to 4 heteroatoms selected from O, S, and N;
each R¹ is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl; wherein each alkyl, cycloalkyl, heterocycloalkyl, aryl, and heteroaryl is independently and optionally substituted with one or more R^{1a};
or two R¹ on the same atom are taken together to form an oxo;
each R^{1a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl; wherein each alkyl, cycloalkyl, heterocycloalkyl, aryl, and heteroaryl is independently and optionally substituted with one or more R;
or two R^{1a} on the same atom are taken together to form an oxo;
n is 0-6;
X is CR^{X} or N;
R^{X} is hydrogen, deuterium, halogen, —CN, —NO₂, —OH, —OR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl;
Y is CR^{Y} or N;
R^{Y} is hydrogen, deuterium, halogen, —CN, —NO₂, —OH, —OR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl;
Z is CR^{Z} or N;
R^{Z} is hydrogen, deuterium, halogen, —CN, —NO₂, —OH, —OR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl;
W is CR^{W} or N;
R^{W} is hydrogen, deuterium, halogen, —CN, —NO₂, —OH, —OR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl;
each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently and optionally substituted with one or more R;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently and optionally substituted with one or more R;
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently and optionally substituted with one or more R;
or R^{c} and R^{d} are taken together with the atom to which they are attached to form a heterocycloalkyl optionally substituted with one or more R; and
each R is independently deuterium, halogen, —CN, —OH, —OC₁-C₆alkyl, —S(═O)C₁-C₆alkyl, —S(═O)₂C₁-C₆alkyl, —S(═O)₂NH₂, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
or two R on the same atom are taken together to form an oxo.

In some embodiments of a compound of Formula (I), Ring A is a 7- to 10-membered cycloalkyl or 7- to 10-membered heterocycloalkyl comprising 1 to 4 heteroatoms selected from O, S, and N. In some embodiments of a compound of Formula (I), Ring A is a 7- to 8-membered cycloalkyl or 7- to 8-membered heterocycloalkyl comprising 1 to 4 heteroatoms selected from O, S, and N.

In some embodiments of a compound of Formula (I), Ring A is a 7- to 10-membered cycloalkyl. In some embodiments of a compound of Formula (I), Ring A is a 7- to 9-membered cycloalkyl. In some embodiments of a compound of Formula (I), Ring A is a 7- to 8-membered cycloalkyl. In some embodiments of a compound of Formula (I), Ring A is a 7-membered cycloalkyl. In some embodiments of a compound of Formula (I), Ring A is a 7- to 10-membered heterocycloalkyl comprising 1 to 4 heteroatoms selected from O, S, and N. In some embodiments of a compound of Formula (I), Ring A is a 7- to 8-membered heterocycloalkyl comprising 1 to 4 heteroatoms selected from O, S, and N. In some embodiments of a compound of Formula (I), Ring A is a 7-membered heterocycloalkyl comprising 1 to 4 heteroatoms selected from O, S, and N. In some embodiments of a compound of Formula (I), the heterocycloalkyl in Ring A comprises 1 to 3 heteroatoms selected from O, S, and N. In some embodiments of a compound of Formula (I), the heterocycloalkyl in Ring A comprises 1 to 3 heteroatoms selected from O and N. In some embodiments of a compound of Formula (I), the heterocycloalkyl in Ring A comprises 1 or 2 heteroatoms selected from O and N. In some embodiments of a compound of Formula (I), the heterocycloalkyl in Ring A comprises 1 heteroatom that is O. In some embodiments of a compound of Formula (I), the heterocycloalkyl in Ring A comprises 1 heteroatom that is N.

In some embodiments of a compound of Formula (I), X is N. In some embodiments of a compound of Formula (I), X is CR^{X}.

In some embodiments of a compound of Formula (I), R^{X} is hydrogen, deuterium, halogen, —CN, —OH, —OR^{a}, —NR^{c}R^{d}, C₁-C₆alkyl, C₁-C₆haloalkyl, or C₁-C₆deuteroalkyl. In some embodiments of a compound of Formula (I), R^{X} is hydrogen, deuterium, halogen, or C₁-C₆alkyl. In some embodiments of a compound of Formula (I), R^{X} is halogen. In some embodiments of a compound of Formula (I), R^{X} is fluoro.

In some embodiments of a compound of Formula (I), Y is N. In some embodiments of a compound of Formula (I), Y is CR^{Z}.

In some embodiments of a compound of Formula (I), R^{Z} is hydrogen, deuterium, halogen, —CN, —OH, —OR^{a}, —NR^{c}R^{d}, C₁-C₆alkyl, C₁-C₆haloalkyl, or C₁-C₆deuteroalkyl. In some embodiments of a compound of Formula (I), R^{X} is hydrogen, deuterium, halogen, —OH, —OR^{a}, or —NR^{c}R^{d}. In some embodiments of a compound of Formula (I), R^{Y} is —OH.

In some embodiments of a compound of Formula (I), Z is N. In some embodiments of a compound of Formula (I), Z is CR^{Z}.

In some embodiments of a compound of Formula (I), R^{Z} is hydrogen, deuterium, halogen, —CN, —OH, —OR^{a}, —NR^{c}R^{d}, C₁-C₆alkyl, C₁-C₆haloalkyl, or C₁-C₆deuteroalkyl. In some embodiments of a compound of Formula (I), R^{Z} is hydrogen.

In some embodiments of a compound of Formula (I), W is N. In some embodiments of a compound of Formula (I), W is CR^{W}.

In some embodiments of a compound of Formula (I), R^{W} is hydrogen, deuterium, halogen, —CN, —OH, —OR^{a}, —NR^{c}R^{d}, C₁-C₆alkyl, C₁-C₆haloalkyl, or C₁-C₆deuteroalkyl. In some embodiments of a compound of Formula (I), R^{W} is hydrogen.

In some embodiments of a compound of Formula (I), each R¹ is independently deuterium, halogen, —CN, —OH, —OR^{a}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl; wherein each alkyl, cycloalkyl, heterocycloalkyl, aryl, and heteroaryl is independently and optionally substituted with one or more R^{1a}; or two R¹ on the same atom are taken together to form an oxo. In some embodiments of a compound of Formula (I), each R¹ is independently deuterium, halogen, —OR^{a}, —NR^{c}R^{d}, —NR^{b}C(═O)NR^{c}R^{d}, —NR^{b}C(═O)R^{a}, —NR^{b}C(═O)OR^{b}, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl; wherein each alkyl is independently and optionally substituted with one or more R^{1a}; or two R¹ on the same atom are taken together to form an oxo. In some embodiments of a compound of Formula (I), each R¹ is independently —OR^{a}, —NR^{c}R^{d}, —NR^{b}C(═O)NR^{c}R^{d}, —NR^{b}C(═O)R^{a}, —NR^{b}C(═O)OR^{b}, C₁-C₆alkyl, or C₁-C₆haloalkyl; wherein each alkyl is independently and optionally substituted with one or more R^{1a}; or two R¹ on the same atom are taken together to form an oxo.

In some embodiments of a compound of Formula (I), each R¹ is independently —OR^{a}, —NR^{c}R^{d}, —NR^{b}C(═O)NR^{c}R^{d}, —NR^{b}C(═O)R^{a}, —NR^{b}C(═O)OR^{b}, C₁-C₆alkyl, or C₁-C₆haloalkyl; wherein each alkyl is independently and optionally substituted with one or more R^{1a}. In some embodiments of a compound of Formula (I), each R¹ is independently —NR^{c}R^{d} or C₁-C₆alkyl optionally substituted with one or more R^{1a}; or two R¹ on the same atom are taken together to form an oxo. In some embodiments of a compound of Formula (I), each R¹ is independently —NR^{c}R^{d} or C₁-C₆alkyl optionally substituted with one or more R^{1a}. In some embodiments of a compound of Formula (I), each R¹ is independently —NR^{c}R^{d}; or two R¹ on the same atom are taken together to form an oxo. In some embodiments of a compound of Formula (I), each R¹ is independently —NR^{c}R^{d}. In some embodiments of a compound of Formula (I), each R¹ is independently C₁-C₆alkyl optionally substituted with one or more R^{1a}; or two R¹ on the same atom are taken together to form an oxo.

In some embodiments of a compound of Formula (I), each R¹ is independently deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently and optionally substituted with one or more R^{1a}. In some embodiments of a compound of Formula (I), each R¹ is independently deuterium, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, cycloalkyl, or heterocycloalkyl; wherein each alkyl, cycloalkyl, and heterocycloalkyl is independently and optionally substituted with one or more R^{1a}. In some embodiments of a compound of Formula (I), each R¹ is independently —OH, —OR^{a}, —NR^{c}R^{d}, C₁-C₆alkyl, C₁-C₆haloalkyl, C₁-C₆aminoalkyl, or heterocycloalkyl; wherein each alkyl, and heterocycloalkyl is independently and optionally substituted with one or more R^{1a}. In some embodiments of a compound of Formula (I), each R¹ is independently —OH, —NR^{c}R^{d}, C₁-C₆alkyl, C₁-C₆aminoalkyl, or heterocycloalkyl; wherein each alkyl, and heterocycloalkyl is independently and optionally substituted with one or more R^{1a}. In some embodiments of a compound of Formula (I), each R¹ is independently C₁-C₆alkyl independently and optionally substituted with one or more R^{1a}. In some embodiments of a compound of Formula (I), each R¹ is independently C₁-C₆alkyl.

In some embodiments of a compound of Formula (I), each R^{1a} is independently deuterium, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently and optionally substituted with one or more R. In some embodiments of a compound of Formula (I), each R^{1a} is independently deuterium, halogen, —CN, —OH, —OR^{a}, —NR^{c}R^{d}, C₁-C₆alkyl, or C₁-C₆haloalkyl; wherein each alkyl is independently and optionally substituted with one or more R. In some embodiments of a compound of Formula (I), each R^{1a} is independently deuterium, halogen or C₁-C₆alkyl.

In some embodiments of a compound of Formula (I), each R^{1a} is independently deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently and optionally substituted with one or more R. In some embodiments of a compound of Formula (I), each R^{1a} is independently deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl; wherein each alkyl is independently and optionally substituted with one or more R. In some embodiments of a compound of Formula (I), each R^{1a} is independently deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl. In some embodiments of a compound of Formula (I), each R^{1a} is independently deuterium, halogen, —CN, —OH, —OR^{a}, —NR^{c}R^{d}, C₁-C₆alkyl, or C₁-C₆haloalkyl. In some embodiments of a compound of Formula (I), each R^{1a} is independently deuterium, halogen, —CN, —OH, —OR^{a}, —NR^{c}R^{d}, or C₁-C₆alkyl. In some embodiments of a compound of Formula (I), each R^{1a} is independently —CN or —NR^{c}R^{d}.

In some embodiments of a compound of Formula (I), n is 1-4. In some embodiments of a compound of Formula (I), n is 1-3. In some embodiments of a compound of Formula (I), n is 1 or 2. In some embodiments of a compound of Formula (I), n is 0 or 1. In some embodiments of a compound of Formula (I), n is 0. In some embodiments of a compound of Formula (I), n is 1. In some embodiments of a compound of Formula (I), n is 2. In some embodiments of a compound of Formula (I), n is 3.

In some embodiments of a compound of Formula (I),

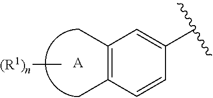

is

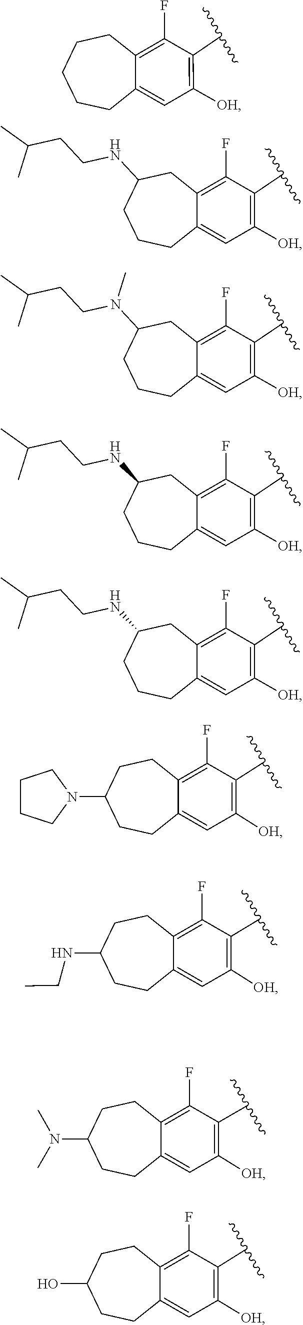

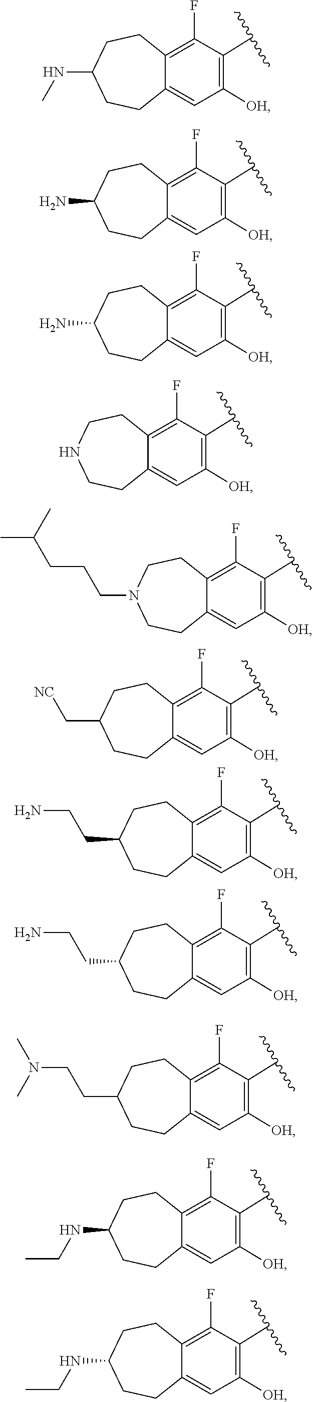

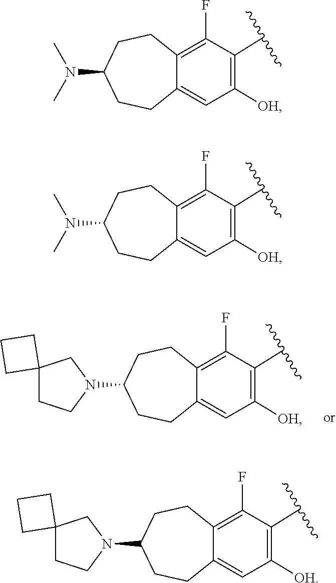

In some embodiments of a compound of Formula (I),

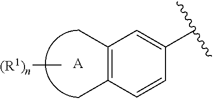

is

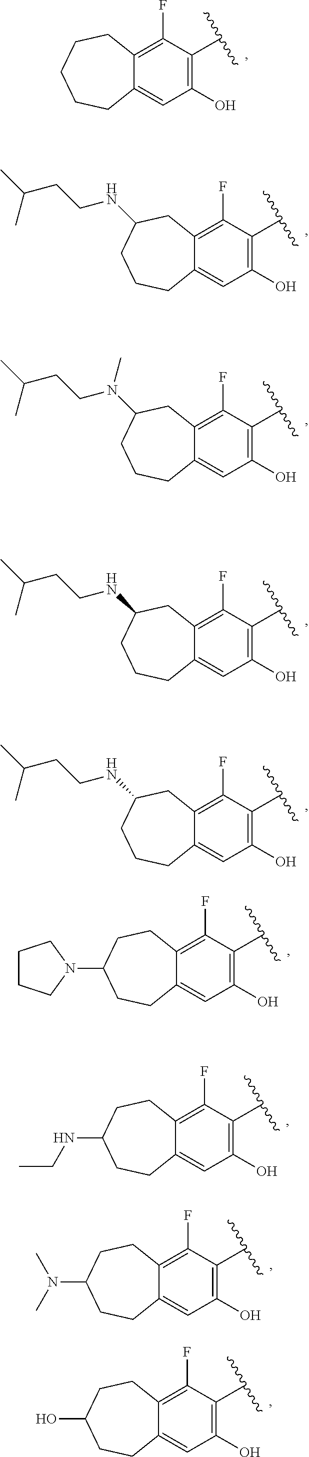

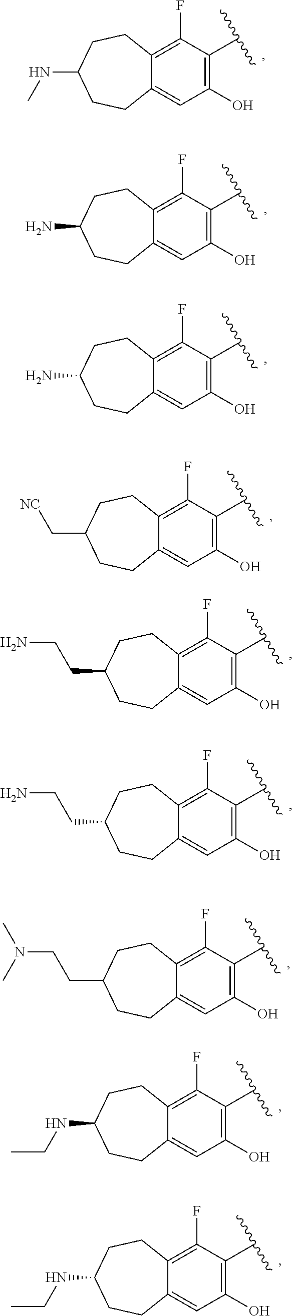

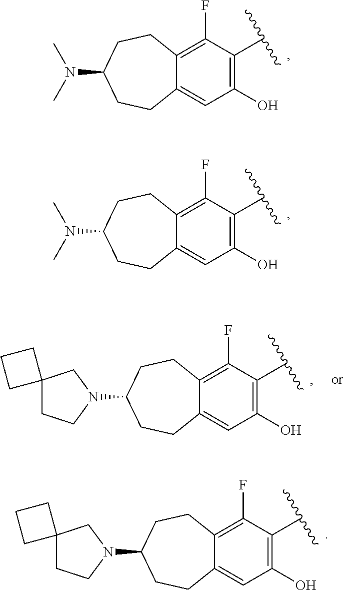 [SMILES: *c1c(O)cc2c(c1F)CC[C@H](N(C)C)CC2 ; *c1c(O)cc2c(c1F)CC[C@@H](N(C)C)CC2 ; *c1c(O)cc2c(c1F)CC[C@@H](N1CCC3(CCC3)C1)CC2 ; *c1c(O)cc2c(c1F)CC[C@H](N1CCC3(CCC3)C1)CC2]

In some embodiments of a compound of Formula (I),

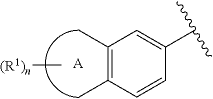

is

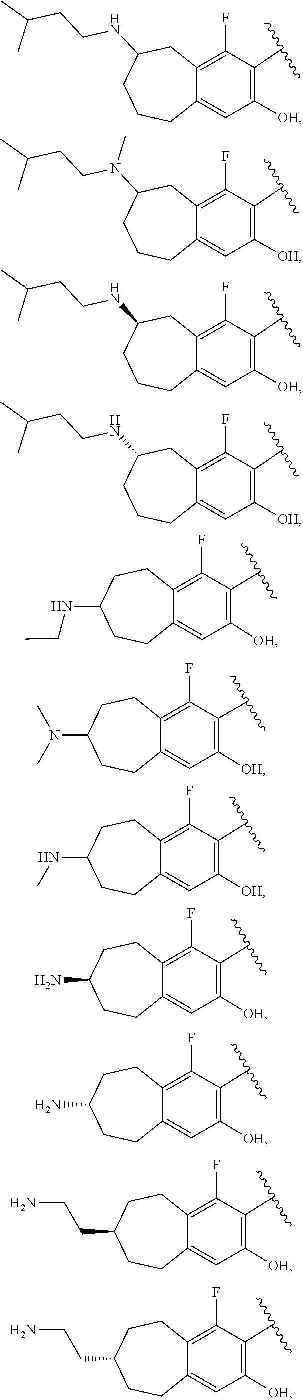

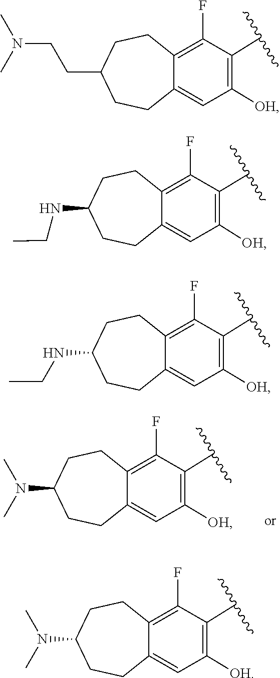

In some embodiments of a compound of Formula (I),

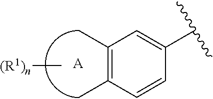

is

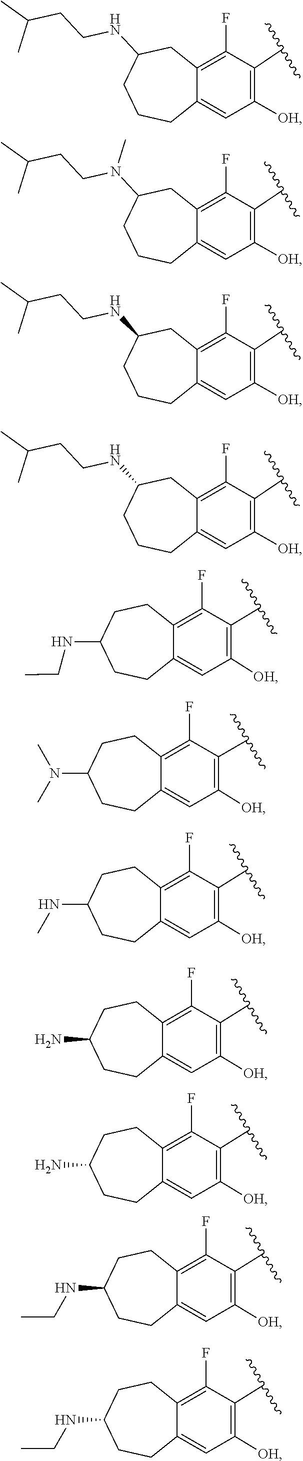

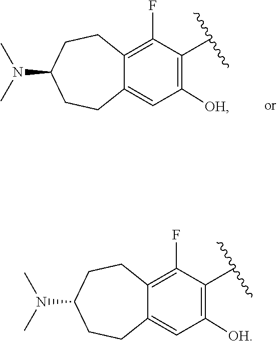 [SMILES: *c1c(O)cc2c(c1F)CC[C@H](N(C)C)CC2 ; "or" ; *c1c(O)cc2c(c1F)CC[C@@H](N(C)C)CC2]

In some embodiments of a compound of Formula (I),

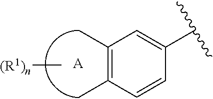

is

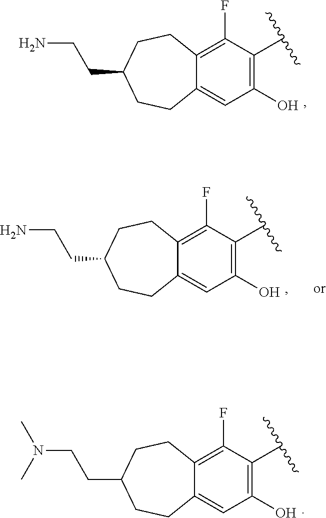 [SMILES: *c1c(O)cc2c(c1F)CC[C@H](CCN)CC2 ; *c1c(O)cc2c(c1F)CC[C@@H](CCN)CC2 ; *c1c(O)cc2c(c1F)CCC(CCN(C)C)CC2]

In some embodiments of a compound of Formula (I),

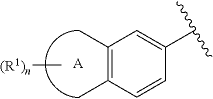

is

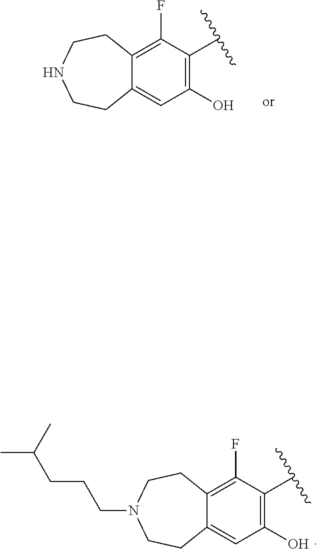 [SMILES: *c1c(O)cc2c(c1F)CCNCC2 ; *c1c(O)cc2c(c1F)CCN(CCCC(C)C)CC2]

In some embodiments of a compound disclosed herein, each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently and optionally substituted with one or more R. In some embodiments of a compound disclosed herein, each R^{a} is independently C₁-C₆alkyl, C₁-C₆haloalkyl, C₁-C₆deuteroalkyl, cycloalkyl, or heterocycloalkyl; wherein each alkyl, cycloalkyl, and heterocycloalkyl is independently and optionally substituted with one or more R. In some embodiments of a compound disclosed herein, each R^{a} is independently C₁-C₆alkyl, C₁-C₆haloalkyl, or cycloalkyl; wherein each alkyl and cycloalkyl is independently and optionally substituted with one or more R. In some embodiments of a compound disclosed herein, each R^{a} is independently C₁-C₆alkyl or cycloalkyl; wherein each alkyl and cycloalkyl is independently and optionally substituted with one or more R. In some embodiments of a compound disclosed herein, each R^{a} is independently C₁-C₆alkyl or C₁-C₆haloalkyl; wherein each alkyl is independently and optionally substituted with one or more R.

In some embodiments of a compound disclosed herein, each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently and optionally substituted with one or more R. In some embodiments of a compound disclosed herein, each R^{b} is independently hydrogen, C₁-C₆alkyl, C₁-C₆haloalkyl, C₁-C₆deuteroalkyl, cycloalkyl, or heterocycloalkyl; wherein each alkyl, cycloalkyl, and heterocycloalkyl is independently and optionally substituted with one or more R. In some embodiments of a compound disclosed herein, each R^{b} is independently hydrogen, C₁-C₆alkyl, C₁-C₆haloalkyl, or cycloalkyl; wherein each alkyl and cycloalkyl is independently and optionally substituted with one or more R. In some embodiments of a compound disclosed herein, each R^{b} is independently hydrogen, C₁-C₆alkyl, or cycloalkyl; wherein each alkyl and cycloalkyl is independently and optionally substituted with one or more R. In some embodiments of a compound disclosed herein, each R^{b} is independently hydrogen, C₁-C₆alkyl, or C₁-C₆haloalkyl; wherein each alkyl is independently and optionally substituted with one or more R. In some embodiments of a compound disclosed herein, each R^{b} is independently hydrogen or C₁-C₆alkyl independently and optionally substituted with one or more R.

In some embodiments of a compound disclosed herein, each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently and optionally substituted with one or more R. In some embodiments of a compound disclosed herein, each R^{c} and R^{d} are independently hydrogen, C₁-C₆alkyl, C₁-C₆haloalkyl, C₁-C₆deuteroalkyl, cycloalkyl, or heterocycloalkyl; wherein each alkyl, cycloalkyl, and heterocycloalkyl is independently and optionally substituted with one or more R. In some embodiments of a compound disclosed herein, each R^{c} and R^{d} are independently hydrogen, C₁-C₆alkyl, C₁-C₆haloalkyl, or cycloalkyl; wherein each alkyl and cycloalkyl is independently and optionally substituted with one or more R. In some embodiments of a compound disclosed herein, each R^{c} and R^{d} are independently hydrogen, C₁-C₆alkyl, or cycloalkyl; wherein each alkyl and cycloalkyl is independently and optionally substituted with one or more R. In some embodiments of a compound disclosed herein, each R^{c} and R^{d} are independently hydrogen, C₁-C₆alkyl, or C₁-C₆haloalkyl; wherein each alkyl is independently and optionally substituted with one or more R. In some embodiments of a compound disclosed herein, each R^{c} and R^{d} are independently hydrogen or C₁-C₆alkyl independently and optionally substituted with one or more R. In some embodiments of a compound disclosed herein, R^{c} is cycloalkyl and R^{d} hydrogen.

In some embodiments of a compound disclosed herein, R^{c} and R^{d} are taken together with the atom to which they are attached to form a heterocycloalkyl optionally substituted with one or more R. In some embodiments of a compound disclosed herein, R^{c} and R^{d} are taken together with the atom to which they are attached to form a heterocycloalkyl.

In some embodiments of a compound disclosed herein, each R is independently deuterium, halogen, —CN, —OH, —OC₁-C₆alkyl, —NH₂, —NHC₁-C₆alkyl, —N(C₁-C₆alkyl)₂, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, or C₁-C₆deuteroalkyl; or two R on the same atom form an oxo. In some embodiments of a compound disclosed herein, each R is independently deuterium, halogen, —CN, —OH, —OC₁-C₆alkyl, —NH₂, —NHC₁-C₆alkyl, —N(C₁-C₆alkyl)₂, C₁-C₆alkyl, C₁-C₆haloalkyl, or C₁-C₆deuteroalkyl; or two R on the same atom form an oxo. In some embodiments of a compound disclosed herein, each R is independently deuterium, halogen, —CN, —OH, —OC₁-C₆alkyl, C₁-C₆alkyl, C₁-C₆haloalkyl, or C₁-C₆deuteroalkyl; or two R on the same atom form an oxo. In some embodiments of a compound disclosed herein, each R is independently deuterium, halogen, —CN, —OH, C₁-C₆alkyl, or C₁-C₆haloalkyl. In some embodiments of a compound disclosed herein, each R is independently halogen, C₁-C₆alkyl, or C₁-C₆haloalkyl. In some embodiments of a compound disclosed herein, each R is independently halogen or C₁-C₆alkyl. In some embodiments of a compound disclosed herein, each R is independently halogen.

Any combination of the groups described above for the various variables is contemplated herein. Throughout the specification, groups and substituents thereof are chosen by one skilled in the field to provide stable moieties and compounds.

In some embodiments, the compound disclosed herein is a compound selected from Table 1, or a pharmaceutically acceptable salt, solvate, or stereoisomer thereof.

TABLE 1

| Example | Structure |
|---|---|
| 1 and 2* | 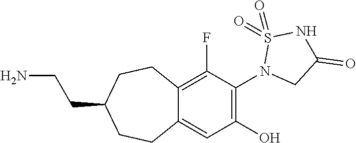 [SMILES: NCC[C@@H]1CCc2cc(O)c(N3CC(=O)NS3(=O)=O)c(F)c2CC1] |
| | 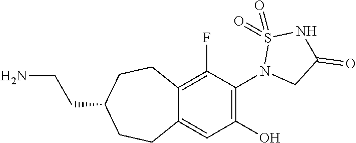 [SMILES: NCC[C@H]1CCc2cc(O)c(N3CC(=O)NS3(=O)=O)c(F)c2CC1] |
| 3 | 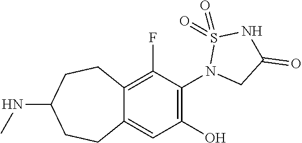 [SMILES: CNC1CCc2cc(O)c(N3CC(=O)NS3(=O)=O)c(F)c2CC1] |
| 4 | 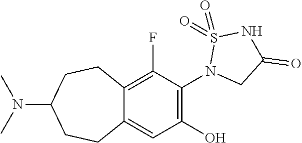 [SMILES: CN(C)C1CCc2cc(O)c(N3CC(=O)NS3(=O)=O)c(F)c2CC1] |
| 5 | 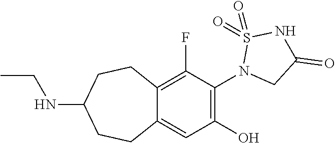 [SMILES: CCNC1CCc2cc(O)c(N3CC(=O)NS3(=O)=O)c(F)c2CC1] |
| 6 and 7* | 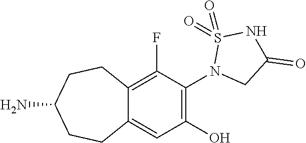 [SMILES: N[C@H]1CCc2cc(O)c(N3CC(=O)NS3(=O)=O)c(F)c2CC1] |
| | 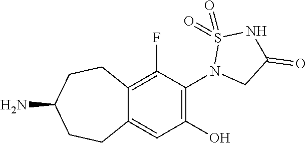 [SMILES: N[C@@H]1CCc2cc(O)c(N3CC(=O)NS3(=O)=O)c(F)c2CC1] |
| 8 | 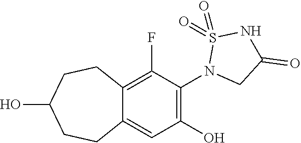 [SMILES: O=C1CN(c2c(O)cc3c(c2F)CCC(O)CC3)S(=O)(=O)N1] |
| 9 | 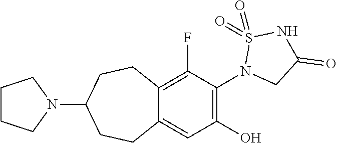 [SMILES: O=C1CN(c2c(O)cc3c(c2F)CCC(N2CCCC2)CC3)S(=O)(=O)N1] |
| 10 and 11* | 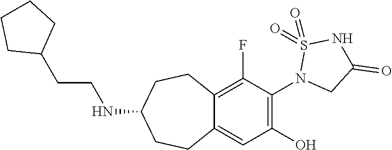 [SMILES: O=C1CN(c2c(O)cc3c(c2F)CC[C@@H](NCCC2CCCC2)CC3)S(=O)(=O)N1] |
| | 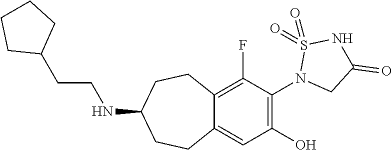 [SMILES: O=C1CN(c2c(O)cc3c(c2F)CC[C@H](NCCC2CCCC2)CC3)S(=O)(=O)N1] |
| 12 | 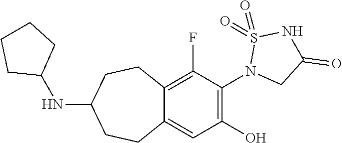 [SMILES: O=C1CN(c2c(O)cc3c(c2F)CCC(NC2CCCC2)CC3)S(=O)(=O)N1] |
| 13 | 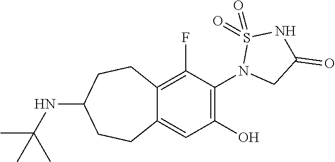 [SMILES: CC(C)(C)NC1CCc2cc(O)c(N3CC(=O)NS3(=O)=O)c(F)c2CC1] |
| 14 and 15* | 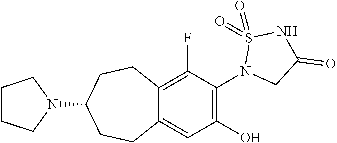 [SMILES: O=C1CN(c2c(O)cc3c(c2F)CC[C@@H](N2CCCC2)CC3)S(=O)(=O)N1] |
| | 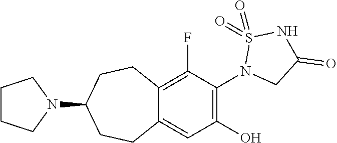 [SMILES: O=C1CN(c2c(O)cc3c(c2F)CC[C@H](N2CCCC2)CC3)S(=O)(=O)N1] |
| 16 and 17* | 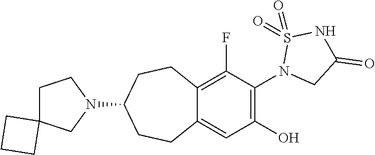 [SMILES: O=C1CN(c2c(O)cc3c(c2F)CC[C@@H](N2CCC4(CCC4)C2)CC3)S(=O)(=O)N1] |
| | 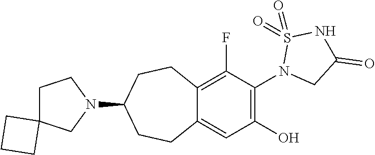 [SMILES: O=C1CN(c2c(O)cc3c(c2F)CC[C@H](N2CCC4(CCC4)C2)CC3)S(=O)(=O)N1] |
| 18 and 19* | 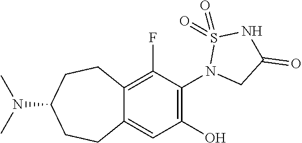 [SMILES: CN(C)[C@H]1CCc2cc(O)c(N3CC(=O)NS3(=O)=O)c(F)c2CC1] |
| | 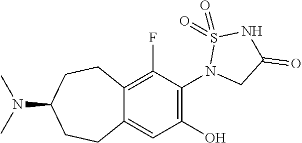 [SMILES: CN(C)[C@@H]1CCc2cc(O)c(N3CC(=O)NS3(=O)=O)c(F)c2CC1] |
| 20 and 21* | 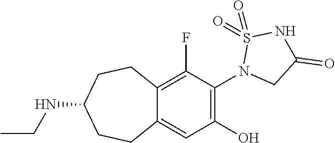 [SMILES: CCN[C@H]1CCc2cc(O)c(N3CC(=O)NS3(=O)=O)c(F)c2CC1] |
| | 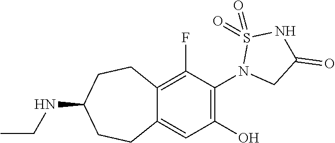 [SMILES: CCN[C@@H]1CCc2cc(O)c(N3CC(=O)NS3(=O)=O)c(F)c2CC1] |
| 22 | 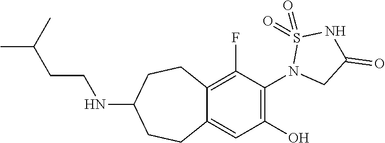 [SMILES: CC(C)CCNC1CCc2cc(O)c(N3CC(=O)NS3(=O)=O)c(F)c2CC1] |
| 23 and 24* | 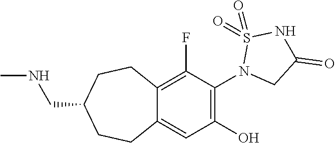 [SMILES: CNC[C@H]1CCc2cc(O)c(N3CC(=O)NS3(=O)=O)c(F)c2CC1] |
| | 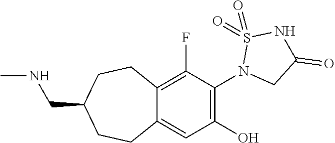 [SMILES: CNC[C@@H]1CCc2cc(O)c(N3CC(=O)NS3(=O)=O)c(F)c2CC1] |
| 25 | 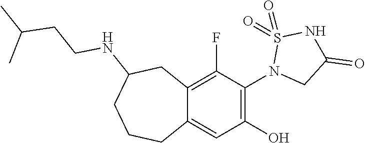 [SMILES: CC(C)CCNC1CCCc2cc(O)c(N3CC(=O)NS3(=O)=O)c(F)c2C1] |
| 26 and 27* | 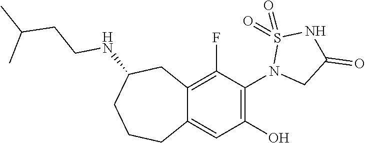 [SMILES: CC(C)CCN[C@H]1CCCc2cc(O)c(N3CC(=O)NS3(=O)=O)c(F)c2C1] |
| | 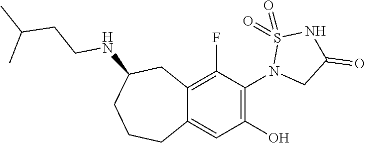 [SMILES: CC(C)CCN[C@@H]1CCCc2cc(O)c(N3CC(=O)NS3(=O)=O)c(F)c2C1] |
| 28 | 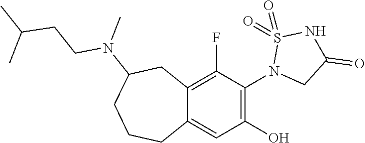 [SMILES: CC(C)CCN(C)C1CCCc2cc(O)c(N3CC(=O)NS3(=O)=O)c(F)c2C1] |
| 29 | 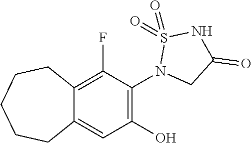 [SMILES: O=C1CN(c2c(O)cc3c(c2F)CCCCC3)S(=O)(=O)N1] |
| 30 | 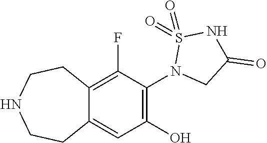 [SMILES: O=C1CN(c2c(O)cc3c(c2F)CCNCC3)S(=O)(=O)N1] |
| 31 | 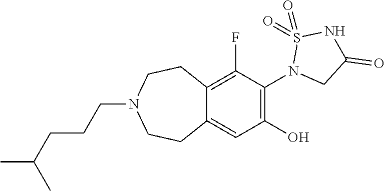 [SMILES: CC(C)CCCN1CCc2cc(O)c(N3CC(=O)NS3(=O)=O)c(F)c2CC1] |
| 32 | 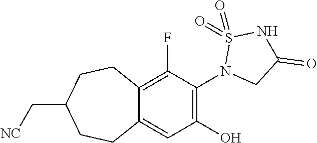 [SMILES: N#CCC1CCc2cc(O)c(N3CC(=O)NS3(=O)=O)c(F)c2CC1] |
| 33 | 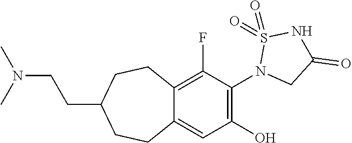 [SMILES: CN(C)CCC1CCc2cc(O)c(N3CC(=O)NS3(=O)=O)c(F)c2CC1] |

*stereochemistry at chiral center was arbitrarily assigned

### Further Forms of Compounds Disclosed Herein

### Isomers/Stereoisomers

In some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, the compounds described herein possess one or more chiral centers and each center exists in the R configuration, or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that would not result in racemization.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that can be incorporated into compounds disclosed herein include isotopes of hydrogen, carbon, nitrogen, oxygen, phosphorous, sulfur, fluorine, and chloride, such as ²H, ³H, ³C, ¹⁴C, ¹⁵N, ¹⁸O, ¹⁷O, ³¹P, ³²P, ³⁵S, ¹⁸F, and ³⁶Cl, respectively. Compounds described herein, and the pharmaceutically acceptable salts, solvates, or stereoisomers thereof which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds disclosed herein, or a solvate, or stereoisomer thereof, or by separately reacting a purified compound in its free form with a suitable acid or base, and isolating the salt thus formed.

Examples of pharmaceutically acceptable salts include those salts prepared by reaction of the compounds described herein with a mineral, organic acid or inorganic base, such salts including, acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate, metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate, undecanoate, and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid and muconic acid. In some embodiments, other acids, such as oxalic, while not in themselves pharmaceutically acceptable, are employed in the preparation of salts useful as intermediates in obtaining the compounds disclosed herein, solvate, or stereoisomer thereof and their pharmaceutically acceptable acid addition salts.

In some embodiments, those compounds described herein which comprise a free acid group react with a suitable base, such as the hydroxide, carbonate, bicarbonate, sulfate, of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine and the like. It should be understood that the compounds described herein also include the quaternization of any basic nitrogen-containing groups they contain. In some embodiments, water or oil-soluble or dispersible products are obtained by such quaternization.

### Solvates

In some embodiments, the compounds described herein exist as solvates. The invention provides for methods of treating diseases by administering such solvates. The invention further provides for methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein can be conveniently prepared or formed during the processes described herein. By way of example only, hydrates of the compounds described herein can be conveniently prepared by recrystallization from an aqueous/organic solvent mixture, using organic solvents including, but not limited to, dioxane, tetrahydrofuran or methanol. In addition, the compounds provided herein can exist in unsolvated as well as solvated forms. In general, the solvated forms are considered equivalent to the unsolvated forms for the purposes of the compounds and methods provided herein.

### Tautomers

In some situations, compounds exist as tautomers. The compounds described herein include all possible tautomers within the formulas described herein. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH.

### Method of Treatment

Disclosed herein are methods of treatment of a disease in which inhibition of PTPN1/PTPN2 is beneficial, the method comprising administering a compound disclosed herein, or a pharmaceutically acceptable salt, solvate, or stereoisomer thereof.

Disclosed herein are methods of treatment of a disease in which inhibition of PTPN1 is beneficial, the method comprising administering a compound disclosed herein, or a pharmaceutically acceptable salt, solvate, or stereoisomer thereof. In some embodiments, the disease in which inhibition of PTPN1 is beneficial is cancer or a metabolic disease.

Disclosed herein are methods of treatment of a disease in which inhibition of PTPN2 is beneficial, the method comprising administering a compound disclosed herein, or a pharmaceutically acceptable salt, solvate, or stereoisomer thereof. In some embodiments, the disease in which inhibition of PTPN2 is beneficial is cancer.

### Cancer

In some embodiments, the compound disclosed herein, or a pharmaceutically acceptable salt, solvate, or stereoisomer thereof, is used to treat cancer.

As used herein, “cancer” refers to human cancers and carcinomas, sarcomas, adenocarcinomas (e.g., papillary adenocarcinomas), lymphomas, leukemias, melanomas, etc., including solid and lymphoid cancers.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound, pharmaceutical composition, or method provided herein include, for example, chronic leukemia, acute nonlymphocytic leukemia, acute lymphocytic leukemia, B-cell chronic lymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, acute myelocytic leukemia, chronic myelocytic leukemia, leukemia cutis, embryonal leukemia, eosinophilic leukemia, erythroleukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocyte leukemia, micromyeloblastic leukemia, monocytic leukemia, myeloblasts leukemia, myelocytic leukemia, myeloid granulocytic leukemia, myelomonocytic leukemia, Naegeli leukemia, plasma cell leukemia, multiple myeloma, plasmacytic leukemia, polycythemia vera, promyelocytic leukemia, Rieder cell leukemia, Schilling's leukemia, stem cell leukemia, subleukemic leukemia, or undifferentiated cell leukemia.

The term “sarcoma” generally refers to a tumor which is made up of a substance like the embryonic connective tissue and is generally composed of closely packed cells embedded in a fibrillar or homogeneous substance. Sarcomas that may be treated with a compound, pharmaceutical composition, or method provided herein include a chondrosarcoma, fibrosarcoma, leiomyosarcoma, lymphosarcoma, lymphangiosarcoma, lymphangioendotheliosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, endotheliosarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, osteogenic sarcoma, parosteal sarcoma, reticulocytic sarcoma, Rous sarcoma, serocystic sarcoma, synovial sarcoma, or telangiectaltic sarcoma.

The term “carcinoma” refers to a malignant new growth made up of epithelial cells tending to infiltrate the surrounding tissues and give rise to metastases. Exemplary carcinomas that may be treated with a compound, pharmaceutical composition, or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bile duct carcinoma, bladder carcinoma, breast carcinoma, Brenner carcinoma, bronchioalveolar carcinoma, bronchiolar carcinoma, bronchiogenic carcinoma, cerebriform carcinoma, cervical carcinoma, cholangiocellular carcinoma, chordoma, chorionic carcinoma, clear cell carcinoma, colloid carcinoma, colon carcinoma, comedo carcinoma, corpus carcinoma, cribriform carcinoma, carcinoma en cuirasse, carcinoma cutaneum, cylindrical carcinoma, cylindrical cell carcinoma, cystadenocarcinoma, duct carcinoma, ductal carcinoma, carcinoma durum, embryonal carcinoma, encephaloid carcinoma, endometrioid carcinoma, epiermoid carcinoma, epithelial carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lobular carcinoma, lung carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, nonpapillary renal cell carcinoma, oat cell carcinoma, carcinoma ossificans, osteoid carcinoma, ovarian carcinoma, pancreatic ductal carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, sebaceous gland carcinoma, seminoma, serous carcinoma, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, sweat gland carcinoma, carcinoma telangiectaticum, carcinoma telangiectodes, transitional cell carcinoma, carcinoma tuberosum, tubular carcinoma, tuberous carcinoma, undifferentiated carcinoma, verrucous carcinoma, or carcinoma villosum.

In some embodiments, the cancer is acoustic neuroma, adrenal cortical cancer, adrenal gland cancer, astrocytoma, benign monoclonal gammopathy, biliary tract cancer, bladder cancer, bone cancer, brain tumor, breast cancer, bronchus cancer, cancer of the hematological tissues, cancer of the hepatic stellate cells, cancer of the oral cavity or pharynx, cancer of the pancreatic stellate cells, carcinoma, central nervous system cancer, cervical cancer, colon cancer, colorectal cancer, craniopharyngioma, ductal carcinoma, endocrine system cancer, endometrial cancer, ependymoma, epithelial ovarian cancer, esophageal cancer, gastric cancer, genitourinary tract cancer, glioblastoma multiforme, glioma, gynecologic cancers, head and neck cancer, hemangioblastoma, Hodgkin's Disease, immunocytic amyloidosis, kidney cancer, laryngeal cancer, leukemia, liver cancer (including hepatocarcinoma), lobular carcinoma, lung cancer, lymphoma, malignant carcinoid, malignant hypercalcemia, malignant pancreatic insulanoma, medullary thyroid cancer, medulloblastoma, melanoma, meningioma, mesothelioma, multiple myeloma, myeloma, neoplasms of the endocrine or exocrine pancreas, neuroblastoma, non-Hodgkin's Lymphoma, oligodendroglioma, oral cancer, ovarian cancer, Paget's Disease of the Nipple, pancreatic cancer, papillary thyroid cancer, peripheral nervous system cancer, Phyllodes Tumors, pinealoma, premalignant skin lesions, primary macroglobulinemia, primary thrombocytosis, prostate cancer, renal cancer, retinoblastoma, rhabdomyosarcoma, salivary gland cancer, sarcoma, skin cancer, small bowel or appendix cancer, stomach cancer, testicular cancer, thyroid cancer, urinary bladder cancer, uterine cancer, Waldenstrom's macroglobulinemia.

### Metabolic Diseases

In some embodiments, the compound disclosed herein, or a pharmaceutically acceptable salt, solvate, or stereoisomer thereof, is used to treat a metabolic disease.

As used herein, the term “metabolic disease” refers to a disease or condition affecting a metabolic process in a subject. Exemplary metabolic diseases include non-alcoholic steatohepatitis (NASH), non-alcoholic fatty liver disease (NAFLD), liver fibrosis, obesity, heart disease, atherosclerosis, arthritis, cystinosis, diabetes (e.g., Type I diabetes, Type II diabetes, or gestational diabetes), metabolic syndrome, phenylketonuria, proliferative retinopathy, or Kearns-Sayre disease. In some embodiments, a compound disclosed herein, is used to treat a metabolic disease (e.g., a metabolic disease described herein) by decreasing or eliminating a symptom of the disease. In some embodiments, the method of treatment comprises decreasing or eliminating a symptom comprising elevated blood pressure, elevated blood sugar level, weight gain, fatigue, blurred vision, abdominal pain, flatulence, constipation, diarrhea, jaundice, and the like.

### Dosing

In certain embodiments, the compositions containing the compound(s) described herein are administered for therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In certain embodiments wherein the patient's condition does not improve, upon the doctor's discretion the administration of the compounds are administered chronically, that is, for an extended period of time, including throughout the duration of the patient's life in order to ameliorate or otherwise control or limit the symptoms of the patient's disease or condition.

In certain embodiments wherein a patient's status does improve, the dose of drug being administered is temporarily reduced or temporarily suspended for a certain length of time (i.e., a “drug holiday”).

Once improvement of the patient's conditions has occurred, a maintenance dose is administered if necessary. Subsequently, in specific embodiments, the dosage, or the frequency of administration, or both, is reduced, as a function of the symptoms.

The amount of a given agent that corresponds to such an amount varies depending upon factors such as the particular compound, disease condition and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated.

In some embodiments, doses employed for adult human treatment are typically in the range of 0.01 mg-5000 mg per day. In some embodiments, the daily dosages appropriate for the compound described herein, or a pharmaceutically acceptable salt thereof, are from about 0.01 to about 50 mg/kg per body weight. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

### Routes of Administration

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a compound as described herein is administered in a local rather than systemic manner, for example, via injection of the compound directly into an organ, often in a depot preparation or sustained release formulation. In specific embodiments, long acting formulations are administered by implantation (for example subcutaneously or intramuscularly) or by intramuscular injection. Furthermore, in other embodiments, the drug is delivered in a targeted drug delivery system, for example, in a liposome coated with organ specific antibody. In such embodiments, the liposomes are targeted to and taken up selectively by the organ. In yet other embodiments, the compound as described herein is provided in the form of a rapid release formulation, in the form of an extended release formulation, or in the form of an intermediate release formulation.

### Pharmaceutical Compositions/Formulations

The compounds described herein are administered to a subject in need thereof, either alone or in combination with pharmaceutically acceptable carriers, excipients, or diluents, in a pharmaceutical composition, according to standard pharmaceutical practice. In some embodiments, the compounds described herein are administered to animals.

In another aspect, provided herein are pharmaceutical compositions comprising a compound described herein, or a pharmaceutically acceptable salt, solvate, or stereoisomer thereof, and at least one pharmaceutically acceptable excipient. Pharmaceutical compositions are formulated in a conventional manner using one or more pharmaceutically acceptable excipients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the pharmaceutically acceptable excipient is selected from carriers, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, and any combinations thereof.

The pharmaceutical formulations described herein include, but are not limited to, aqueous liquid dispersions, liquids, gels, syrups, elixirs, slurries, suspensions, self-emulsifying dispersions, solid solutions, liposomal dispersions, aerosols, solid oral dosage forms, powders, immediate release formulations, controlled release formulations, fast melt formulations, tablets, capsules, pills, powders, dragees, effervescent formulations, lyophilized formulations, delayed release formulations, extended release formulations, pulsatile release formulations, multiparticulate formulations, and mixed immediate and controlled release formulations.

### Combination

Disclosed herein are methods of treating cancer using a compound disclosed herein, or a pharmaceutically acceptable salt, solvate, or stereoisomer thereof, in combination with an additional therapeutic agent.

In some embodiments, the additional therapeutic agent is an anticancer agent.

In some embodiments, the additional therapeutic agent is administered at the same time as the compound disclosed herein. In some embodiments, the additional therapeutic agent and the compound disclosed herein are administered sequentially. In some embodiments, the additional therapeutic agent is administered less frequently than the compound disclosed herein. In some embodiments, the additional therapeutic agent is administered more frequently than the compound disclosed herein. In some embodiments, the additional therapeutic agent is administered prior than the administration of the compound disclosed herein. In some embodiments, the additional therapeutic agent is administered after the administration of the compound disclosed herein.

In some embodiments, the additional therapeutic agent is an immunotherapeutic agent. In some embodiments, the immunotherapeutic agent is an anti-PD-1 antibody, an anti-PD-L1 antibody, or an anti-CTLA-4 antibody.

### EXAMPLES

### Example 1 and Example 2: (R)-5-(7-(2-aminoethyl)-1-fluoro-3-hydroxy-6,7,8,9-tetrahydro-5H-benzo[7]annulen-2-yl)-1,2,5-thiadiazolidin-3-one 1,1-dioxide and (S)-5-(7-(2-aminoethyl)-1-fluoro-3-hydroxy-6,7,8,9-tetrahydro-5H-benzo[7]annulen-2-yl)-1,2,5-thiadiazolidin-3-one 1,1-dioxide

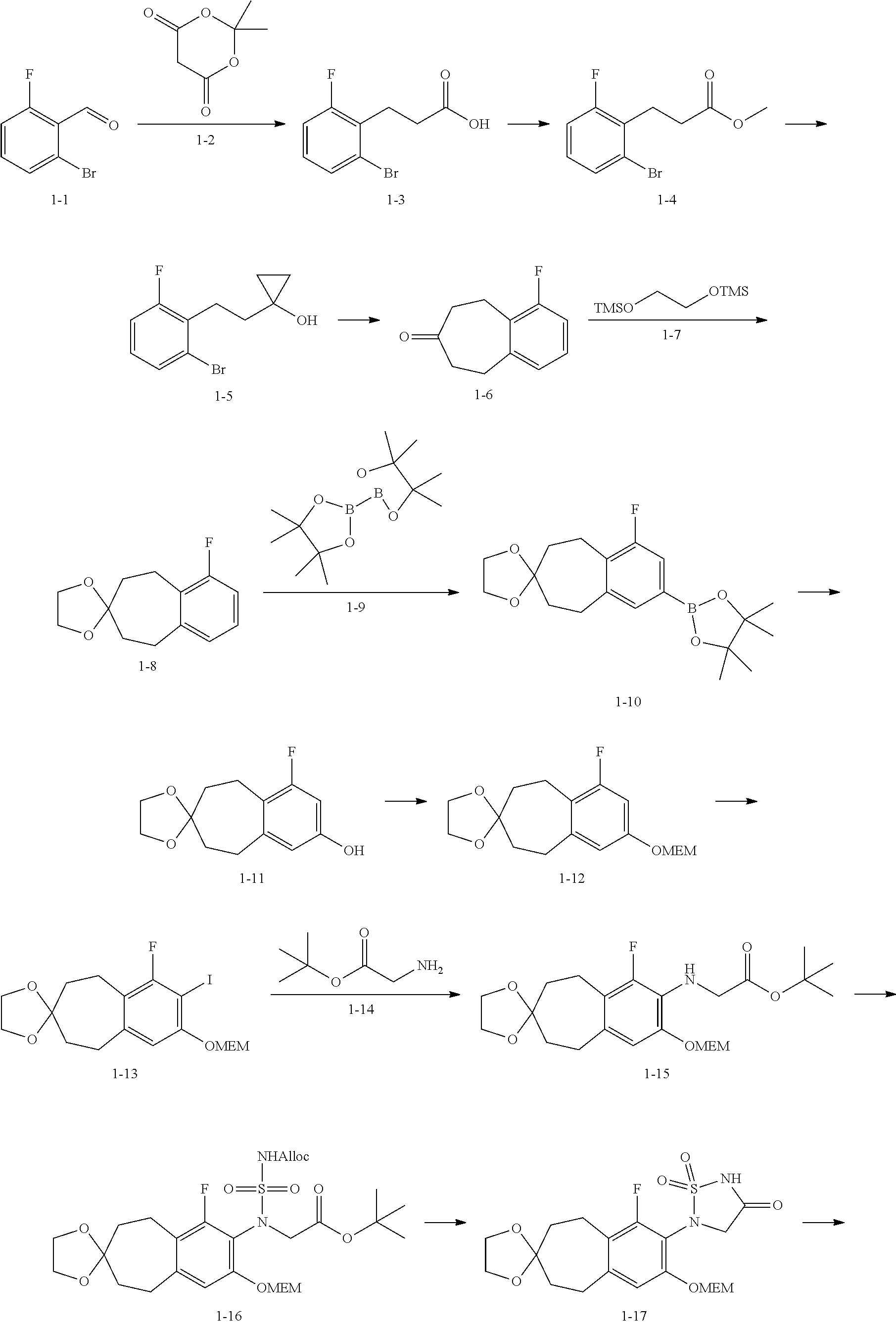

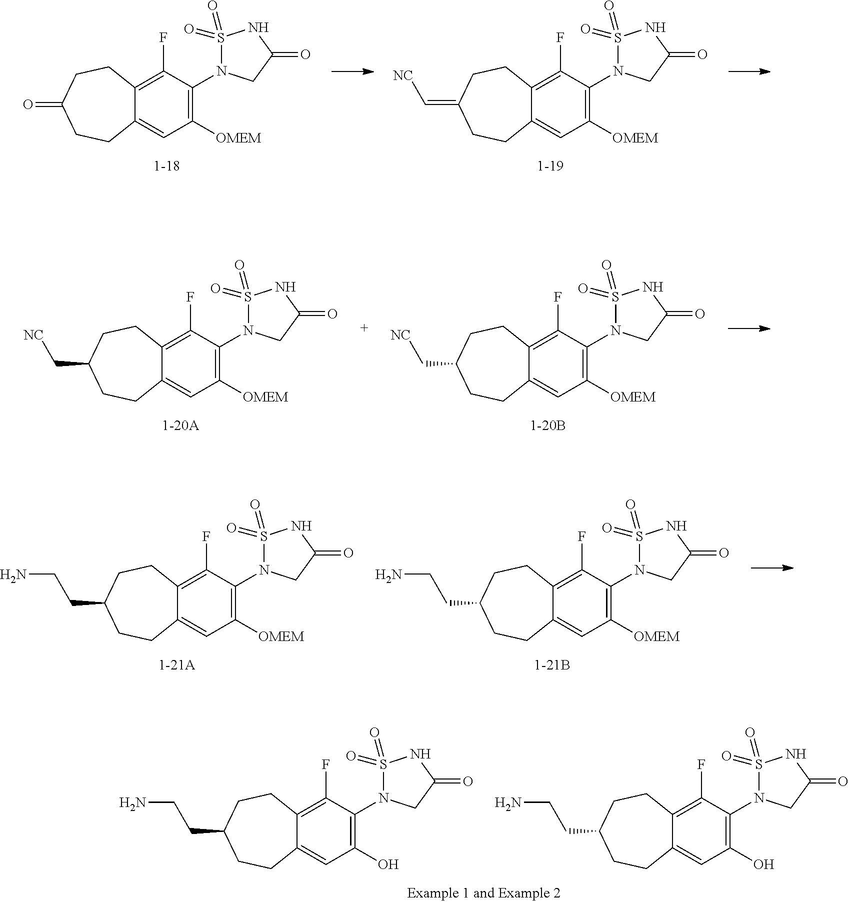

### Step 1: General Procedure of Compound 1-3

To a container with formic acid (378 g, 7.8 mol, 6.4 eq) was added triethylamine (398.7 g, 3.9 mol, 548 mL, 3.2 eq) at 0° C. To this was then added 1-1 (250 g, 1.23 mol, 1 eq) and 1-2 (177.4 g, 1.2 mol, 1 eq) then heated to 100° C. and allowed to stir for 12 h. The reaction mixture was cooled to ambient temperature and then ice H₂O was added (˜1 L). The result suspension was acidified with 6M HCl to adjust pH=1. The precipitated crystal was filtered then washed with water (500 mL×3). The precipitate was then resuspended in chloroform (500 mL) and dried with Na₂SO₄. The mixture was then filtered and concentrated under reduced pressure to give 1-3 (377 g, 1.53 mol, 61% yield) as a pale solid. ¹H NMR (400 MHz, CDCl₃) δ 7.35 (d, J=7.8 Hz, 1H) 7.09 (m, 1H) 6.98-7.04 (m, 1H) 3.13-3.17 (m, 2H) 2.61-2.68 (in, 2H).

### Step 2: General Procedure of Compound 1-4

A solution of 1-3 (370 g, 1.50 mol, 1 eq) in MeOH (1 L) at 0° C. was stirred for 5 min. To this was then added SOCl₂ (534 g, 4.4 mol, 325 mL, 3 eq) dropwise maintaining the temperature at 0° C., then was allowed to stir for 2 h. The resulting reaction mixture was filtered and concentrated under reduced pressure to give a crude. The crude was purified by column chromatography (SiO₂, petroleum ether/ethyl acetate=1/0 to 10/1) to give 1-4 (300 g, 1.15 mol, 76% yield) as a brown oil. ¹H NMR (400 MHz, CDCl₃) δ 7.31 (d, J=7.8 Hz, 1H) 7.01-7.08 (m, 1H) 6.94-7.00 (m, 1H) 3.68 (s, 3H) 3.11 (m, 2H) 2.53-2.60 (in, 2H).

### Step 3: General Procedure of Compound 1-5

A solution of 1-4 (100 g, 383 mmol, 1 eq) and Ti(Oi-Pr)₄ (152 g, 536 mmol, 158 mL, 1.4 eq) in THF (1 L) was cooled to 0° C. and allowed to stir for 20 min. To this was then added 3M EtMgBr (510 mL, 4 eq) at 0° C. and then allowed to stir for an additional 12 h. The resulting reaction mixture was quenched by addition of sat. NH₄Cl (aq) (2 L) then was extracted with ethyl acetate (500 mL×4). The combined organic layers were washed with brine (1 L), dried over Na₂SO₄, then filtered and concentrated under reduced pressure to give a 1-5 (50 g, 192 mmol, 50% yield) as a white solid. ¹H NMR (400 MHz, CDCl₃) δ 7.11 (d, J=7.8 Hz, 1H) 6.74-6.85 (m, 2H) 2.84 (td, J=7.75, 2.25 Hz, 2H) 1.67-1.82 (m, 1H) 1.57-1.63 (m, 2H) 0.50-0.57 (m, 2H) 0.16-0.23 (m, 2H).

### Step 4: General Procedure of Compound 1-6

To a solution of 1-5 (30 g, 115.78 mmol, 1 eq) in acetonitrile (300 mL) was added Pd(OAc)₂ (2.60 g, 11.5 mmol, 0.1 eq), K₃PO₄ (49.1 g, 231 mmol, 2 eq) and DPPB (9.88 g, 23 mmol, 0.2 eq) then heated to 80° C. and allowed to stir for 12 h under N₂. The resulting reaction mixture was diluted with H₂O (150 mL) then extracted with ethyl acetate (100 mL×3). The combined organic layers were washed with brine (100 mL), dried over Na₂SO₄, then filtered and concentrated under reduced pressure to give a crude. The crude was then purified by column chromatography (SiO₂, petroleum ether/ethyl acetate=1/0 to 10/1) to give 1-6 (9.1 g, 51 mmol, 44% yield) as a brown oil. ¹H NMR (400 MHz, CDCl₃) δ 7.10-7.18 (m, 1H) 6.94-7.03 (m, 2H) 2.88-3.00 (m, 4H) 2.60 (m, 4H).

### Step 5: General Procedure of Compound 1-8

To a solution of 1-6 (10.7 g, 60 mmol, 1 eq) and 1-7 (18.5 g, 90 mmol, 1.5 eq) in CH₂Cl₂ (200 mL) was added TMSOTf (2.6 g, 12 mmol, 2.1 mL, 0.2 eq) at 25° C. and allowed to stir for 12 h. The resulting reaction mixture was quenched by addition H₂O (100 mL) then was extracted with ethyl acetate (100 mL×3). The combined organic layers were washed with brine (50 mL×3), dried over Na₂SO₄, then filtered and concentrated under reduced pressure to give a crude. The crude was purified by column chromatography (SiO₂, petroleum ether/ethyl acetate=1/0 to 10/1) to give 1-8 (11 g, 49 mmol, 82% yield) as a brown oil. ¹H NMR (400 MHz, CDCl₃) δ 7.07 (m, 1H) 6.87-6.96 (m, 2H) 4.03 (s, 4H) 2.79-2.93 (m, 4H) 1.82 (br d, J=8.0 Hz, 4H).

### Step 6: General Procedure of Compound 1-10

To a solution of 1-8 (5 g, 22 mmol, 1 eq) in THF (50 mL) was added 1-9 (11.4 g, 44 mmol, 2 eq), chloroiridium; (1Z,5Z)-cycloocta-1,5-diene (1.5 g, 2.2 mmol, 0.1 eq), 4-tert-butyl-2-(4-tert-butyl-2-pyridyl) and pyridine (1.8 g, 6.7 mmol, 0.3 eq) then heated to 70° C. and allowed to stir for 12 h under N₂. The resulting reaction mixture was cooled to 25° C. and then quenched by adding H₂O (1 L) slowly. To this was then added brine (400 mL) then extracted with ethyl acetate (200 mL×3). The combined organic phases were dried over Na₂SO₄, then filtered and concentrated under reduced pressure to afford a yellow oil. The crude was purified by column chromatography (SiO₂, petroleum ether/ethyl acetate=1/0 to 10/1) to give (30 g, crude) as a pale oil. ¹H NMR (400 MHz, CDCl₃) δ 7.13-7.43 (m, 2H) 3.91 (s, 4H) 2.69-2.83 (m, 4H) 1.62-1.74 (m, 4H) 1.22-1.28 (m, 12H).

### Step 7: General Procedure of Compound 1-11

To a solution of 1-10 (32 g, 91 mmol, 1 eq) in acetone (300 mL) was added a solution of oxone (112 g, 183 mmol, 2.0 eq) in H₂O (300 mL) at 0° C. dropwise over 15 min. This was then stirred for an additional 10 min. The resulting reaction mixture was quenched by addition H₂O (1 L) then extracted with ethyl acetate (500 mL×3). The combined organic layers were washed with brine (100 mL×3), dried over Na₂SO₄, then filtered and concentrated under reduced pressure to give a crude. The crude was then purified by column chromatography (SiO₂, petroleum ether/ethyl acetate=1/0 to 3/1) to give 1-11 (41 g, 172 mmol, 93% yield) as white solid. ¹H NMR (400 MHz, CDCl₃) δ 6.69-6.81 (m, 1H) 6.38-6.44 (m, 1H) 4.02 (d, J=2.3 Hz, 4H) 2.67-2.91 (m, 4H) 1.79 (br d, J=8.6 Hz, 4H) 1.24-1.30 (m, 4H).

### Step 8: General Procedure of Compound 1-12

To a solution of 1-11 (41 g, 172 mmol, 1 eq) in THF (300 mL) was added NaH (13.7 g, 344 mmol, 60% purity, 2 eq) at 0° C. and allowed to stir for 0.5 h under N₂. To this was then added MEMCl (32.1 g, 258 mmol, 29.4 mL, 1.5 eq) dropwise. The reaction mixture was allowed to warm to 25° C. and stirred for 12 h. The resulting suspension was quenched with a mixture of sat. NH₄Cl(aq) and 1M Na₂S₂O₃(aq) (1:1, 600 mL). The reaction mixture was stirred for 5 minutes at 25° C. then was extracted with ethyl acetate (330 mL×3). The combined organic layers were washed with H₂O (600 mL) and brine (600 mL), dried over Na₂SO₄, then filtered and concentrated under reduced pressure to give a crude. The crude was then purified by column chromatography (SiO₂, petroleum ether/ethyl acetate=1/0 to 3/1) to give 1-12 (31 g, 94 mmol, 68% yield) as a colorless oil. ¹H NMR (400 MHz, CDCl₃) δ 6.71-6.91 (m, 1H) 6.53-6.60 (m, 1H) 5.12-5.20 (m, 2H) 3.92 (s, 4H) 3.77-3.82 (m, 1H) 3.70-3.76 (m, 1H) 3.44-3.50 (m, 2H) 3.27-3.31 (m, 3H) 2.63-2.81 (m, 4H) 1.70 (br d, J=7.7 Hz, 4H).

### Step 9: General Procedure of Compound 1-13

To a solution of 2,2,6,6-tetramethylpiperidine (2.3 g, 16 mmol, 2.8 mL, 2 eq) in THF (20 mL) was added 2.5 M n-BuLi (6.6 mL, 2 eq) at 0° C. and allowed to stir for 0.5 h under N₂. The reaction mixture was then cooled to −78° C. and then was added a solution of 1-12 (2.7 g, 8.2 mmol, 1 eq) in THF (10 mL) followed by TMEDA (1.4 g, 12.4 mmol, 1.87 mL, 1.5 eq) and allowed to stir for an additional 2 h. To this was then added a solution of I₂ (5.2 g, 20 mmol, 4.17 mL, 2.5 eq) in THF (5 mL) and allowed to stir for an additional 1 h. The resulting suspension was quenched with a mixture of sat. NH₄Cl(aq) and 1M Na₂S₂O₃(aq) (1:1, 30 mL) and allowed to stir for 5 minutes at 25° C. The resulting reaction mixture was then extracted with ethyl acetate (10 mL×2). The combined organic layers were washed with water (20 mL) and brine (20 mL), dried over Na₂SO₄, then filtered and concentrated under reduced pressure to give a crude. The crude was then purified by column chromatography (SiO₂, petroleum ether/ethyl acetate=1/0 to 3/1) to give 1-13 (2.8 g, 6.19 mmol, 74% yield) as a colorless oil. ¹H NMR (400 MHz, CDCl₃) δ 6.77 (s, 1H), 5.32 (s, 2H), 4.01 (s, 4H), 3.90-3.85 (m, 2H), 3.60-3.55 (m, 2H), 3.39 (s, 3H), 2.88-2.76 (m, 4H), 1.83-1.73 (m, 4H).

### Step 10: General Procedure of Compound 1-15

To a solution of 1-13 (1 g, 2.2 mmol, 1 eq) in 1,4-dioxane (10 mL) was added 1-14 (435 mg, 3.3 mmol, 1.5 eq), Cs₂CO₃, (2.1 g, 6.6 mmol, 3 eq), XPhos (210 mg, 442 μmol, 0.2 eq) and BrettPhos Pd G₃ (200 mg, 221 μmol, 0.1 eq) then heated to 100° C. and allowed to stir for 48 h. The resulting suspension was quenched with a mixture of sat. NH₄Cl(aq) and 1M Na₂S₂O₃(aq) (1:1, 200 mL) and allowed to stir for 5 minutes at 25° C. The resulting reaction mixture was then extracted with ethyl acetate (100 mL×5). The combined organic layers were washed with water (300 mL) and brine (200 mL), dried over Na₂SO₄, then filtered and concentrated under reduced pressure to give a crude. The crude was then purified by column chromatography (SiO₂, petroleum ether/ethyl acetate=1/0 to 3/1) to give 1-15 (16.8 g, 36 mmol, 61% yield) as a white solid. ¹H NMR (400 MHz, CDCl₃) δ 6.73 (s, 1H) 5.24-5.31 (m, 2H) 4.00 (s, 4H) 3.95 (s, 2H) 3.86 (m, 2H) 3.58 (m, 2H) 3.39 (s, 3H) 2.67-2.79 (m, 4H) 1.72-1.80 (m, 4H) 1.44 (s, 9H).

### Step 11: General Procedure of Compound 1-16

To a solution of N-(oxomethylene)sulfamoyl chloride (1.9 g, 13.8 mmol, 1.2 mL, 1.5 eq) in CH₂Cl₂ (30 mL) was added prop-2-en-1-ol (1.65 g, 28.4 mmol, 1.93 mL, 3.08 eq) at 0° C. and allowed to stir for 0.5 h. To this was then added a solution of 1-15 (4.2 g, 9.2 mmol, 1 eq) and DIPEA (2.38 g, 18.4 mmol, 3.2 mL, 2 eq) in CH₂Cl₂ (30 mL) at 25° C. and allowed to stir for 1 h. To the resulting reaction mixture was added H₂O (150 mL) then extracted with CH₂Cl₂ (50 mL×2). The combined organic layers were washed with brine (100 mL), dried over Na₂SO₄, then filtered and concentrated under reduced pressure to give a crude. The crude was then purified by column chromatography (SiO₂, petroleum ether/ethyl acetate=1/0 to 3/1) to give 1-16 (17.8 g, 28 mmol, 78% yield) as a white solid. ¹H NMR (400 MHz, CDCl₃) δ 8.37 (s, 1H) 6.84 (s, 1H) 5.24-5.41 (m, 6H) 4.64-4.70 (m, 3H) 4.22 (d, J=17.8 Hz, 1H) 4.00 (s, 4H) 3.86 (m, 2H) 3.51-3.59 (m, 2H) 3.33 (s, 3H) 2.79 (br s, 4H) 1.69-1.86 (m, 4H) 1.42 (s, 9H).

### Step 12: General Procedure of Compound 1-17

To a solution of 1-16 (1 g, 1.62 mmol, 1 eq) in MeOH (10 mL) was added NaOMe (1.46 g, 8.08 mmol, 30% purity, 5 eq) and Pd(PPh₃)₄ (186 mg, 161 μmol, 0.1 eq) then heated to 60° C. and allowed to stir for 1 h. The resulting reaction mixture was then filtered and concentrated under reduced pressure to give 1-17 (13.05 g, crude) as a white solid. ¹H NMR (400 MHz, CDCl₃) δ 6.87 (s, 1H) 5.31 (s, 2H) 4.35-4.44 (m, 2H) 4.01 (s, 4H) 3.80-3.92 (m, 3H) 3.46-3.61 (m, 3H) 3.35-3.40 (m, 3H) 2.79 (br s, 4H) 1.72-1.85 (m, 4H).

### Step 13: General Procedure of Compound 1-18

A solution of 1-17 (0.8 g, 1.74 mmol, 1 eq) in formic acid (8 mL) was allowed to stir for 2 h at 25° C. The reaction mixture was filtered and concentrated under reduced pressure to give a residue. The crude product was used into the next step without further purification. 1-18 (800 mg, crude) was obtained as a yellow solid. LCMS (ESI−): m/z=415.2 (M−H)⁻.

### Step 14: General Procedure of Compound 1-19

To a solution of 2-diethoxyphosphorylacetonitrile (957 mg, 5.4 mmol, 874 μL, 1.5 eq) in THF (10 mL) was added NaH (216 mg, 5.4 mmol, 60% purity, 1.5 eq) and allowed to stir for 0.5 h at 0° C. under N₂. To this was then added a solution of 1-18 (1.5 g, 3.6 mmol, 1 eq) in THF (5 mL) and stirred for an additional 2 h at 25° C. The resulting suspension was quenched with a mixture of sat. NH₄Cl(aq) and 1M Na₂S₂O₃(aq) (1:1, 10 mL) and allowed to stir for 5 minutes at 25° C. The resulting reaction mixture was then extracted with ethyl acetate (10 mL×2). The combined organic layers were washed with water (10 mL) and brine (10 mL), dried over Na₂SO₄, then filtered and concentrated under reduced pressure to give 1-19 (2 g, crude) as yellow oil.

### Step 15: General Procedure of Compound 1-20

To a solution of 1-19 (1.5 g, 3.41 mmol, 1 eq) in MeOH (10 mL) was added Pd/C (0.7 g, 10% purity) then purged with H₂ (15 psi). The reaction mixture was then heated to 40° C. and allowed to stir for 12 h. The resulting reaction mixture was then filtered over celite and concentrated under reduced pressure to give a crude. The crude was then purified by prep-HPLC to give 1-20 (0.2 g, 453 μmol, 13% yield) as a white solid. 1-20A and 1-20B was obtained by SFC separation of 1-20 (stereochemistry at chiral center was arbitrarily assigned).

### Step 16: General Procedure of Compound 1-21

To a solution of 1-20A or 1-20B (40 mg, 90 μmol, 1 eq) in EtOH (2 mL) was added NH₃—H₂O (12 mg, 90 μmol, 13 μL, 26% purity, 1 eq) and Raney-Ni (0.02 g) under N₂ atmosphere. The reaction mixture was purged with H₂ (50 psi) then heated to 40° C. and allowed to stir for 12 h. The resulting reaction mixture was then filtered over celite and concentrated under reduced pressure to give 1-21A or 1-21B (40 mg, crude) as a yellow solid.

### Step 17: General Procedure of Example 1 and 2

A solution of 1-21A (20 mg, 44 μmol, 1 eq) in 4M HCl/dioxane (1 mL, 89 eq), was allowed to stir for 1 h at 20° C. The reaction mixture was then concentrated under reduced pressure to give a crude. The crude was then purified by prep-HPLC to give Example 1 (5.8 mg, 16 μmol, 36% yield). LCMS (ESI−): m/z=356.1 (M−H)⁻. ¹H NMR (400 MHz, DMSO-d₆) δ 9.88-9.61 (m, 1H), 7.77 (br s, 2H), 6.55 (s, 1H), 4.22 (s, 2H), 3.04-2.92 (m, 1H), 2.88-2.76 (m, 2H), 2.76-2.60 (m, 2H), 2.37 (br t, J=13.3 Hz, 1H), 1.95-1.81 (m, 2H), 1.71 (br d, J=6.3 Hz, 1H), 1.55-1.42 (m, 2H), 1.11-0.88 (m, 2H).

Similarly, example 2 was obtained: LCMS (ESI−): m/z=356.0 (M−H)⁻. ¹H NMR (400 MHz, DMSO-d₆) δ 6.49 (s, 1H) 3.91 (s, 2H) 2.93-3.03 (m, 1H) 2.75-2.88 (m, 2H) 2.64-2.71 (m, 2H) 2.41 (s, 1H) 1.86 (br dd, J=10.9, 3.3 Hz, 2H) 1.63-1.76 (m, 1H) 1.39-1.52 (m, 2H) 0.91-1.11 (m, 2H).

### Example 3-Example 22: Compounds were Prepared According to the Procedures Described in 1-22 Using the Appropriate Amines

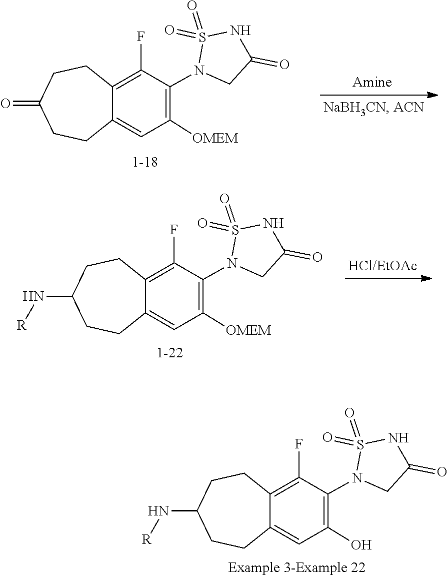

### Step 1: General Procedure of Compound 1-22

To a solution of 1-18 (100 mg, 240 μmol, 1 eq) in acetonitrile (1 mL) was added corresponding amine (1 eq) and NaBH₃CN (45 mg, 720 μmol, 12 μL, 3 eq) and allowed to stir for 12 h at 25° C. The resulting reaction mixture was then concentrated under reduced pressure to give a crude. The crude was then purified by HPLC to give 1-22. Separation of enantiomers was obtained by SFC separation of 1-22.

### Step 2: General Procedure of Example 3-Example 22

A solution of 1-22 (1 eq) in 4 M HCl/ethyl acetate (1 mL) was allowed to stir for 2 h at 25° C. The reaction mixture was then concentrated under reduced pressure to give a crude. The crude was purified by HPLC (column: Waters Xbridge BEH C18 100×30 mm×10 um; mobile phase: [A: H₂O (NH₄HCO₃), B: acetonitrile]; B: 1-15%, 8 min) to give Example 3-Example 22.

| Example | LCMS | ¹H NMR |
|---|---|---|
| 3 | LCMS (ESI−): | ¹H NMR (400 MHz, D₂O) δ 6.68 (s, 1H), 4.34 (s, 2H), 3.43-3.32 |
| | m/z = 341.8 | (m, 1H), 3.25-3.15 (m, 1H), 2.90-2.72 (m, 2H), 2.69 (s, 3H), 2.47- |
| | (M − H)⁻ | 2.36 (m, 1H), 2.35-2.25 (m, 2H), 1.51-1.33 (m, 2H) |
| 4 | LCMS (ESI−): | ¹H NMR (400 MHz, CD₃OD) δ 6.64 (s, 1H) 4.43 (s, 2H) 3.51-3.62 |
| | m/z = 356.1 | (m, 1H) 2.82-2.90 (m, 2H) 2.81 (s, 6H) 2.27-2.46 (m, 4H) 1.49- |
| | (M − H)⁻ | 1.60 (m, 2H) |
| 5 | LCMS (ESI+): | ¹H NMR (400 MHz, CD₃OD) δ 6.62 (s, 1H) 4.39-4.44 (m, 2H) |
| | m/z = 358.3 | 3.21-3.28 (m, 1H) 3.10-3.16 (m, 2H) 2.82-2.87 (m, 2H) 2.40-2.52 |
| | (M − H)⁻ | (m, 2H) 2.37 (br d, J = 7.2 Hz, 2H) 1.35-1.44 (m, 2H) 1.32 (t, J = |
| | | 7.2 Hz, 3H) |
| 6 | LCMS (ESI−): | ¹H NMR (400 MHz, D₂O) δ 7.47 (s, 1H), 6.67 (s, 1H), 4.33 (s, 2H), |
| | m/z = 328.1 | 3.56-3.41 (m, 1H), 3.24-3.12 (m, 1H), 2.85-2.76 (m, 2H), 2.52-2.36 |
| | (M − H)⁻ | (m, 1H), 2.29-2.15 (m, 2H), 1.52-1.34 (m, 2H) |
| 7 | LCMS (ESI−): | ¹H NMR (400 MHz, D₂O) δ 6.67 (s, 1H), 4.33 (s, 2H), 3.55-3.44 |
| | m/z = 328.1 | (m, 1H), 3.23-3.12 (m, 1H), 2.86-2.74 (m, 2H), 2.48-2.35 (m, 1H), |
| | (M − H)⁻ | 2.30-2.15 (m, 2H), 1.53-1.32 (m, 2H) |
| 8 | LCMS (ESI−): | ¹H NMR (400 MHz, D₂O) δ 6.65 (s, 1H), 4.33 (s, 2H), 3.92 (br s, |
| | m/z = 329.0 | 1H), 3.02 (br dd, J = 8.7, 14.8 Hz, 1H), 2.86-2.70 (m, 1H), 2.68- |
| | (M − H)⁻ | 2.54 (m, 1H), 2.38 (br t, J = 12.8 Hz, 1H), 2.03 (br s, 2H), 1.37 (br |
| | | dd, J = 10.0, 19.9 Hz, 2H) |
| 9 | LCMS (ESI+): | ¹H NMR (400 MHz, CD₃OD) δ 6.60 (s, 1H), 4.23 (s, 1H), 3.52 (br |
| | m/z = 384.1 | s, 1H), 3.42-3.34 (m, 2H), 3.28-3.20 (m, 2H), 2.88-2.74 (m, 2H), |
| | (M + H)⁺ | 2.46-2.32 (m, 3H), 2.05 (br s, 4H), 1.58-1.40 (m, 2H), 1.29 (br d, J = |
| | | 3.2 Hz, 1H) |
| 10 | LCMS (ESI+): | ¹H NMR (400 MHz, DMSO-d₆) δ 9.80 (dt, J = 5.0, 2.3 Hz, 1 H) |
| | m/z = 424.2 | 8.38 (br d, J = 2.8 Hz, 2 H) 6.57 (s, 1 H) 4.19 (s, 2 H) 3.35 (br dd, J = |
| | (M + H)⁺ | 11.6, 8.0 Hz, 1 H) 2.98-3.10 (m, 1 H) 2.86-2.96 (m, 2 H) 2.67- |
| | | 2.81 (m, 2 H) 2.37 (br dd, J = 14.4, 13.0 Hz, 1 H) 2.19-2.30 (m, 2 |
| | | H) 1.68-1.82 (m, 3 H) 1.53-1.63 (m, 4 H) 1.43-1.52 (m, 2 H) 1.22- |
| | | 1.36 (m, 2 H) 1.01-1.13 (m, 2 H) |
| 11 | LCMS (ESI−): | ¹H NMR (400 MHz, DMSO-d₆) δ 9.94 (br d, J = 5.15 Hz, 1 H) |
| | m/z = 424.2 | 8.46 (br d, J = 4.39 Hz, 2 H) 6.58 (s, 1 H) 4.26 (s, 2 H) 3.31-3.39 |
| | (M − H)⁻ | (m, 1 H) 3.04 (br dd, J = 15.3, 7.5 Hz, 1 H) 2.91 (br d, J = 2.7 Hz, 2 |
| | | H) 2.68-2.84 (m, 2 H) 2.34-2.42 (m, 1 H) 2.21-2.30 (m, 2 H) 1.69- |
| | | 1.82 (m, 3 H) 1.53-1.66 (m, 4 H) 1.43-1.53 (m, 2 H) 1.21-1.38 (m, |
| | | 2 H) 1.02-1.13 (m, 2 H) |
| 12 | LCMS (ESI+): | ¹H NMR (400 MHz, DMSO-d₆) δ 9.12 (br s, 1H), 8.14 (br s, 1H), |
| | m/z = 398.2 | 6.54 (s, 1H), 3.92 (s, 2H), 3.64 (br s, 1H), 3.42-3.33 (m, 1H), 3.04 |
| | (M + H)⁺ | (br dd, J = 7.2, 15.1 Hz, 1H), 2.74 (br s, 2H), 2.45-2.36 (m, 1H), |
| | | 2.34-2.19 (m, 2H), 2.00 (br s, 2H), 1.71 (br s, 2H), 1.55 (br s, 4H), |
| | | 1.38-1.17 (m, 2H) |
| 13 | LCMS (ESI−): | ¹H NMR (400 MHz, DMSO-d₆) δ 9.13 (s, 1H), 7.95 (br s, 2H), 6.54 |
| | m/z = 384.1 | (s, 1H), 3.92 (s, 2H), 3.63 (br s, 1H), 3.03 (br dd, J = 7.3, 14.8 Hz, |
| | (M − H)⁻ | 1H), 2.87-2.76 (m, 1H), 2.75-2.69 (m, 1H), 2.47-2.41 (m, 1H), 2.16 |
| | | (br s, 2H), 1.52-1.37 (m, 2H), 1.34 (s, 9H) |
| 14 | LCMS (ESI−): | ¹H NMR (400 MHz, D₂O) δ 10.36 (br s, 1H), 10.16-9.85 (m, 1H), |
| | m/z = 382.1 | 6.61 (s, 1H), 4.33-4.24 (m, 2H), 3.55-3.32 (m, 3H), 3.22-2.98 (m, |
| | (M − H)⁻ | 3H), 2.87-2.63 (m, 2H), 2.45-2.24 (m, 3H), 2.00-1.78 (m, 4H), |
| | | 1.56-1.35 (m, 2H) |
| 15 | LCMS (ESI−): | ¹H NMR (400 MHz, DMSO-d₆) δ 10.45 (br s, 1H), 10.10-9.86 (m, |
| | m/z = 382.1 | 1H), 6.69-6.56 (m, 1H), 4.34-4.21 (m, 2H), 3.44 (br s, 3H), 3.20- |
| | (M − H)⁻ | 3.01 (m, 3H), 2.88-2.67 (m, 2H), 2.44-2.26 (m, 3H), 2.03-1.78 (m, |
| | | 4H), 1.61-1.36 (m, 2H) |
| 16 | LCMS (ESI+): | ¹H NMR (400 MHz, D₂O) δ 6.66 (s, 1H), 4.31 (s, 2H), 3.62-3.33 |
| | m/z = 424.2 | (m, 3H), 3.29-3.08 (m, 3H), 2.91-2.59 (m, 2H), 2.30 (br d, J = 10.9 |
| | (M + H)⁺ | Hz, 3H), 2.20-1.96 (m, 5H), 1.95-1.70 (m, 3H), 1.53-1.28 (m, 2H) |
| 17 | LCMS (ESI+): | ¹H NMR (400 MHz, D₂O) δ 6.67 (s, 1H), 4.32 (s, 2H), 3.59-3.39 |
| | m/z = 424.2 | (m, 3H), 3.30-3.09 (m, 3H), 2.89-2.64 (m, 2H), 2.43-2.23 (m, 3H), |
| | (M + H)⁺ | 2.19-1.96 (m, 5H), 1.95-1.73 (m, 3H), 1.54-1.28 (m, 2H) |
| 18 | LCMS (ESI+): | ¹H NMR (400 MHz, CD₃OD) δ 6.63 (d, J = 1.0 Hz, 1 H) 4.42 (s, 2 |
| | m/z = 358.1 | H) 3.56 (m, 1 H) 2.83-2.90 (m, 2 H) 2.82 (s, 6 H) 2.24-2.47 (m, 4 |
| | (M + H)⁺ | H) 1.47-1.62 (m, 2 H) |
| 19 | LCMS (ESI+): | ¹H NMR (400 MHz, CD₃OD) δ 6.64 (s, 1 H) 4.43 (s, 2 H) 3.51- |
| | m/z = 358.1 | 3.62 (m, 1 H) 2.82-2.90 (m, 2 H) 2.81 (s, 6 H) 2.27-2.46 (m, 4 H) |
| | (M + H)⁺ | 1.49-1.60 (m, 2 H) |
| 20 | LCMS (ESI+): | ¹H NMR (400 MHz, CD₃OD) δ 6.62 (s, 1 H) 4.41 (s, 2 H) 3.23- |
| | m/z = 358.1 | 3.28 (m, 1 H) 3.13 (d, J = 7.2 Hz, 2 H) 2.84 (br t, J = 4.6 Hz, 2 H) |
| | (M + H)⁺ | 2.32-2.51 (m, 4 H) 1.35-1.44 (m, 2 H) 1.32 (t, J = 7.25 Hz, 3 H) |
| 21 | LCMS (ESI+): | ¹H NMR (400 MHz, CD₃OD) δ 6.62 (s, 1 H) 4.39-4.44 (m, 2 H) |
| | m/z = 358.1 | 3.21-3.28 (m, 1 H) 3.10-3.16 (m, 2 H) 2.82-2.87 (m, 2 H) 2.40-2.52 |
| | (M + H)⁺ | (m, 2 H) 2.37 (br d, J = 7.2 Hz, 2 H) 1.35-1.44 (m, 2 H) 1.32 (t, J = |
| | | 7.2 Hz, 3 H) |
| 22 | LCMS (ESI−): | ¹H NMR (400 MHz, D₂O) δ 6.68 (s, 1H), 4.34 (s, 2H), 3.48-3.33 |
| | m/z = 398.2 | (m, 1H), 3.26-3.15 (m, 1H), 3.11-3.02 (m, 2H), 2.85-2.77 (m, 2H), |
| | (M − H)⁻ | 2.46-2.27 (m, 3H), 1.68-1.58 (m, 1H), 1.57-1.47 (m, 2H), 1.46-1.32 |
| | | (m, 2H), 0.89 (d, J = 6.6 Hz, 6H) |

### Example 23 and example 24: (S)-5-(1-fluoro-3-hydroxy-7-((methylamino)methyl)-6,7,8,9-tetrahydro-5H-benzo[7]annulen-2-yl)-1,2,5-thiadiazolidin-3-one 1,1-dioxide and (R)-5-(1-fluoro-3-hydroxy-7-((methylamino)methyl)-6,7,8,9-tetrahydro-5H-benzo[7]annulen-2-yl)-1,2,5-thiadiazolidin-3-one 1,1-dioxide

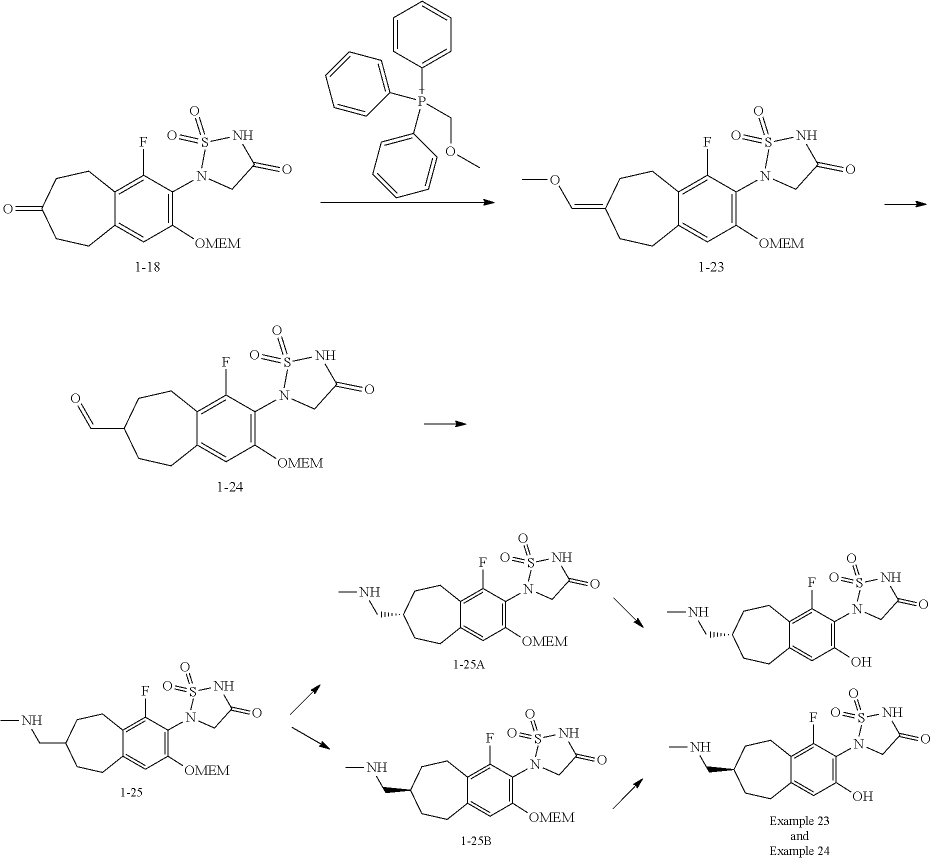

### Step 1: General Procedure of Compound 1-23

To a solution of methoxymethyl(triphenyl)phosphonium; chloride (1.98 g, 5.76 mmol, 1.5 eq) in THF (15 mL) was added t-BuOK (1 M, 11.53 mL, 3 eq) at 0° C. and allowed to stir for 0.5 h under N₂. To this was then added 1-18 (1.6 g, 3.84 mmol, 1 eq) and allowed to warm to 25° C. and stirred for an additional 0.5 h. To the reaction mixture was added H₂O (15 mL) and then extracted with ethyl acetate (5 mL×3). The combined organic layers were washed with brine (10 mL), dried over Na₂SO₄, filtered, then concentrated under reduced pressure to give a crude. The crude was then purified by column chromatography (SiO₂, ethyl acetate/methanol=1/0 to 3/1) to give 1-23 (1 g, 2.25 mmol, 58% yield) as a yellow solid. ¹H NMR (400 MHz, CD₃OD) δ 6.89 (d, J=4.38 Hz, 1H) 5.91 (d, J=2.50 Hz, 1H) 5.28 (d, J=1.13 Hz, 2H) 4.26 (s, 2H) 3.81-3.88 (m, 2H) 3.56 (br d, J=5.13 Hz, 2H) 3.33 (s, 3H) 2.76 (br dd, J=10.44, 4.94 Hz, 4H) 2.28-2.39 (m, 2H) 2.05-2.16 (m, 2H).

### Step 2: General Procedure of Compound 1-24

To a solution of 1-23 (0.3 g, 674 μmol, 1 eq) in CH₂Cl₂ (2 mL) was added TFA (837 mg, 7.3 mmol, 545 μL, 10.8 eq) and allowed to stir for 15 min at 0° C. The reaction mixture was then concentrated under reduced pressure to give 1-24 (0.9 g, crude) as a yellow solid.

### Step 3: General Procedure of Compound 1-25

To a solution of 2M methylamine (33.8 mL, 145 eq) in CH₂Cl₂ (2 mL) was added 1-24 (0.2 g, 464 μmol, 1 eq) and diisopropylethylamine (120 mg, 929 μmol, 161 μL, 2 eq) and allowed to stir for 0.5 h at 25° C. To this was then added NaBH₃CN (58 mg, 929 μmol, 2 eq) and allowed to stir for an additional 0.5 h. To the resulting reaction mixture was added 1M HCl (0.5 mL) and H₂O (15 mL) then extracted with CH₂Cl₂ (5 mL×2). The combined organic layers were washed with brine (10 mL), dried over Na₂SO₄, then filtered and concentrated under reduced pressure to give a crude.

### Step 4: General Procedure of Compound 1-25A and 1-25B

The crude was purified by HPLC to give 1-25 (0.02 g, 44 μmol, 9% yield) as a yellow solid. 1-25A and 1-25B were obtained by SFC separation of 1-25 (stereochemistry at chiral center was arbitrarily assigned). ¹H NMR (400 MHz, CD₃OD) δ 6.89 (s, 1H) 5.28 (s, 2H) 4.23 (s, 2H) 3.82-3.88 (m, 2H) 3.52-3.58 (m, 2H) 3.33 (s, 3H) 3.20 (br dd, J=14.88, 7.0 Hz, 1H) 2.80-2.92 (m, 4H) 2.72 (s, 3H) 2.43-2.55 (m, 1H) 1.95-2.15 (m, 3H) 1.06-1.23 (m, 2H).

### Step 5: General Procedure of Example 23 and 24

A solution of 1-25A (20 mg, 44 μmol, 1 eq) in 4M HCl/EtOAc (1 mL) was allowed to stir for 0.5 h at 20° C. The reaction mixture was then concentrated under reduce pressure to give a crude product. The crude product was triturated with ethyl acetate (0.5 mL) to give Example 23 (5 mg, 12 μmol, 26% yield) as a white solid. LCMS (ESI+): m/z=358.1 (M+H)⁺. ¹H NMR (400 MHz, DMSO-d₆) δ 9.28 (br d, J=1.8 Hz, 1H), 8.25 (br d, J=1.8 Hz, 2H), 6.53 (s, 1H), 4.02 (s, 2H), 2.99 (br dd, J=14.5, 6.9 Hz, 1H), 2.77-2.84 (m, 2H), 2.72 (br d, J=5.9 Hz, 2H), 2.54-2.58 (m, 3H), 2.35-2.45 (m, 1H), 1.86-2.03 (m, 3H), 1.00-1.14 (m, 2H).

Example 24: LCMS (ESI+): m/z=358.1 (M+H)⁺. ¹H NMR (400 MHz, DMSO-d₆) δ 9.88 (br d, J=1.88 Hz, 1H), 8.36 (br d, J=1.8 Hz, 2H), 6.55 (s, 1H), 4.22 (s, 2H), 2.97 (br dd, J=14.5, 6.9 Hz, 1H), 2.71-2.80 (m, 4H), 2.54-2.56 (m, 4H), 1.90-1.98 (m, 3H), 1.05-1.08 (m, 2H).

### Example 25: 5-(1-fluoro-3-hydroxy-8-(isopentylamino)-6,7,8,9-tetrahydro-5H-benzo[7]annulen-2-yl)-1,2,5-thiadiazolidin-3-one 1,1-dioxide

### Example 26 and example 27: (S)-5-(1-fluoro-3-hydroxy-8-(isopentylamino)-6,7,8,9-tetrahydro-5H-benzo[7]annulen-2-yl)-1,2,5-thiadiazolidin-3-one 1,1-dioxide and (R)-5-(1-fluoro-3-hydroxy-8-(isopentylamino)-6,7,8,9-tetrahydro-5H-benzo[7]annulen-2-yl)-1,2,5-thiadiazolidin-3-one 1,1-dioxide

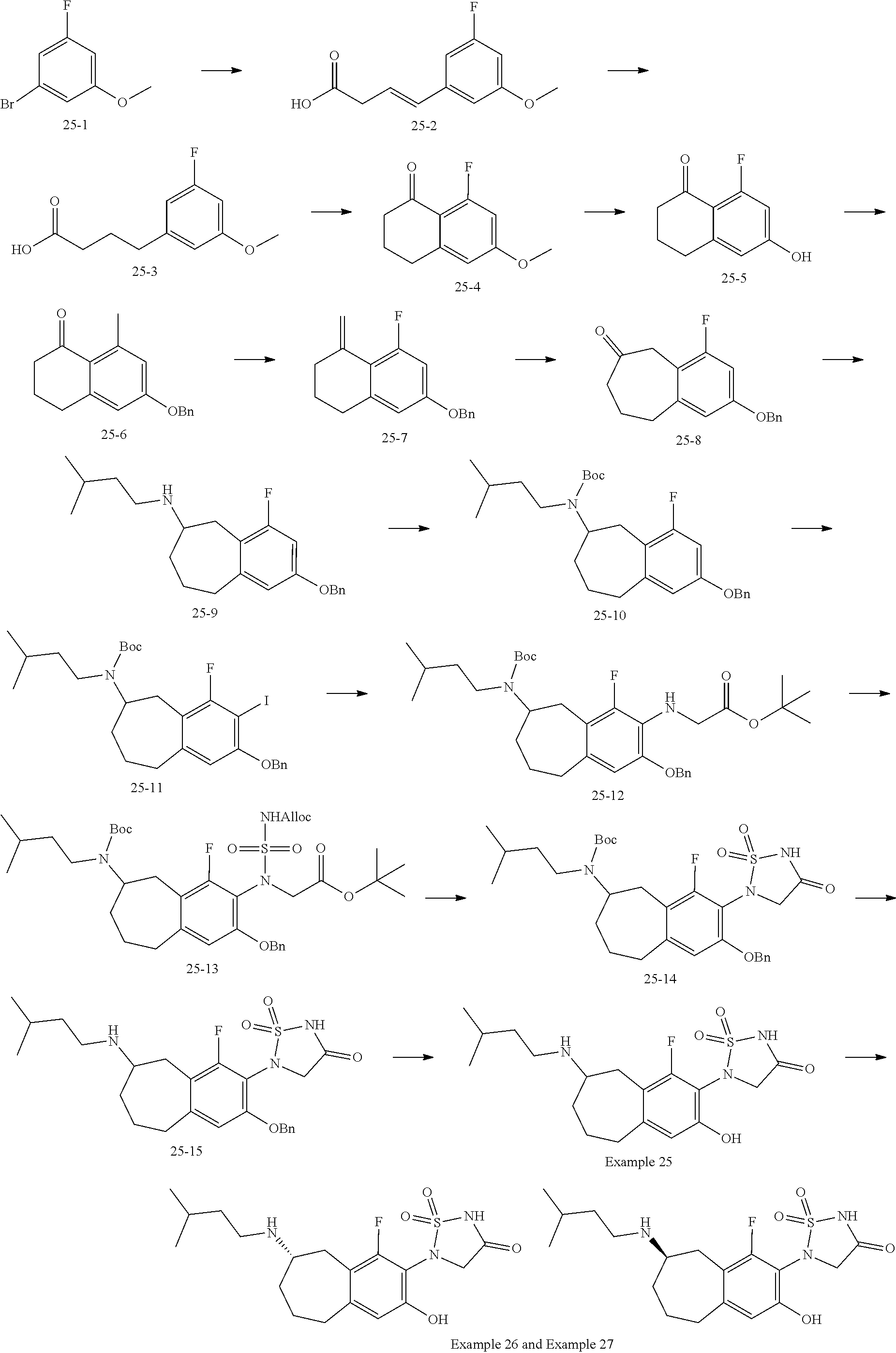

### Step 1: General Procedure of Compound 25-2

To a solution of 25-1 (20 g, 97.55 mmol, 1 eq), but-3-enoic acid (20.6 g, 239 mmol, 2.46 eq), tris-o-tolylphosphane (5.94 g, 19.5 mmol, 0.2 eq) and triethylamine (49.3 g, 487 mmol, 67 mL, 5 eq) in DMA (200 mL) was added Pd(OAc)₂ (2.19 g, 9.75 mmol, 0.1 eq) the heated to 120° C. and allowed to stir for 12 h under N₂. To the resulting reaction mixture was then added brine (400 mL) then extracted with ethyl acetate (300 mL×3). The combined organic layers were washed with brine (800 mL×5), dried over Na₂SO₄, then filtered and concentrated under reduced pressure to give a crude. The crude was then purified by column chromatography (SiO₂, petroleum ether/ethyl acetate=2/1 to 0/1) to give 25-2 (18.1 g, 86.11 mmol, 88% yield) as a yellow oil. ¹H NMR (400 MHz, CD₃OD) δ 6.77-6.70 (m, 2H), 6.55 (td, J=2.3, 10.6 Hz, 1H), 6.49-6.42 (m, 1H), 6.40-6.30 (m, 1H), 3.80 (s, 3H), 3.23 (dd, J=1.1, 6.9 Hz, 2H).

### Step 2: General Procedure of Compound 25-3

To a solution of 25-2 (18.1 g, 86 mmol, 1 eq) in THF (300 mL) was added Pd/C (6 g, 10% purity) under N₂. The reaction mixture was then purged with H₂ (50 psi) heated to 50° C. and allowed to stir for 12 h. The reaction mixture was then filtered and concentrated under reduced pressure to give 25-3 (14.2 g, 66.91 mmol, 77% yield) as yellow oil. ¹H NMR (400 MHz, CD₃OD) δ 6.59 (s, 1H), 6.55-6.47 (m, 2H), 3.77 (s, 3H), 2.62 (t, J=7.6 Hz, 2H), 2.30 (t, J=7.3 Hz, 2H), 1.89 (quin, J=7.5 Hz, 2H).

### Step 3: General Procedure of Compound 25-4

A solution of 25-3 (14.2 g, 66 mmol, 1 eq) in PPA (71 g) was heated to 100° C. and allowed to stir for 5 h. To the resulting reaction mixture was then added sat. NaOH (aq) (500 mL) and stirred for an additional 5 min. The aqueous phase was then extracted with ethyl acetate (400 mL×3). The combined organic phase was washed with brine (1 L), dried with Na₂SO₄, then filtered and concentrated under reduced pressure to give a crude. The crude was then purified by column chromatography (SiO₂, petroleum ether/ethyl acetate=4/1 to 2/1) to give 25-4 (4.86 g, 25 mmol, 37% yield) as a yellow solid. ¹H NMR (400 MHz, CD₃OD) δ 6.69 (s, 1H), 6.60 (dd, J=2.4, 13.5 Hz, 1H), 3.86 (s, 3H), 2.96 (t, J=6.0 Hz, 2H), 2.62-2.52 (m, 2H), 2.06 (quin, J=6.3 Hz, 2H).

### Step 4: General Procedure of Compound 25-5

To a solution of 25-4 (4.7 g, 24.20 mmol, 1 eq) in toluene (47 mL) was added AlCl₃ (9.68 g, 72.6 mmol, 3.97 mL, 3 eq) then heated to 110° C. and allowed to stir for 30 min. The reaction mixture was then poured into ice water (w/w=1/1, 40 mL) and stirred for an additional 5 min. The aqueous phase was then extracted with ethyl acetate (25 mL×3). The combined organic phase was washed with brine (30 mL), dried with Na₂SO₄, then filtered and concentrated under reduced pressure to give a crude. The crude was then purified by prep-TLC (SiO₂, petroleum ether/ethyl acetate=1/1, Rf0.3) to give 25-5 (3.5 g, 19.4 mmol, 80% yield) as a yellow solid.

### Step 5: General Procedure of Compound 25-6

To a solution of 25-5 (2.1 g, 11.6 mmol, 1 eq) in DMF (20 mL) wad added BnBr (2.39 g, 13.9 mmol, 1.66 mL, 1.2 eq) and K₂CO₃ (3.22 g, 23 mmol, 2 eq) and allowed to stir for 2 h at 25° C. To the resulting reaction mixture was then added H₂O (30 mL) then extracted with ethyl acetate (20 mL×3). The combined organic layers were washed with brine (15 mL×3), dried with Na₂SO₄, then filtered and concentrated under reduced pressure to give a crude. The crude was then purified by column chromatography (SiO₂, petroleum ether/ethyl acetate=50/1 to 25/1) to give 25-6 (3 g, 11.10 mmol, 95% yield) as a yellow oil.

### Step 6: General Procedure of Compound 25-7

To a solution of 25-6 (4 g, 14.8 mmol, 1 eq) in THF (40 mL) was added methyl(triphenyl)phosphonium bromide (12.6 g, 35.52 mmol, 2.4 eq) then 1M t-BuOK (35.5 mL, 2.4 eq) dropwise then allowed to stir at 0.5 h at 20° C. under N₂. To the resulting reaction mixture was added H₂O (30 mL) then extracted with ethyl acetate (20 mL×3). The combined organic layers were washed with brine (45 mL), dried with Na₂SO₄, then filtered and concentrated under reduced pressure to give a crude. The crude was then purified by prep-TLC (SiO₂, petroleum ether/ethyl acetate=10/1, Rf=0.4) to give 25-7 (3.9 g, 14.5 mmol, 98% yield) as a yellow oil. ¹H NMR (400 MHz, CDCl₃) δ 7.50-7.31 (m, 5H), 6.61 (d, J=1.9 Hz, 1H), 6.52 (dd, J=2.5, 13.7 Hz, 1H), 5.77-5.67 (m, 1H), 5.04 (s, 2H), 2.67 (t, J=7.6 Hz, 2H), 2.21-2.10 (m, 5H).

### Step 7: General Procedure of Compound 25-8

To a solution of 25-7 (2.9 g, 10.8 mmol, 1 eq) in MeOH (45 mL) was added [hydroxy(phenyl)-A3-iodanyl]4-methylbenzenesulfonate (4.24 g, 10.81 mmol, 1 eq) and allowed to stir for 1 h at 20° C. The reaction mixture was then concentrated under reduced pressure to give a crude. The crude was then purified by column chromatography (SiO₂, petroleum ether/ethyl acetate=24/1) to give 25-8 (1.5 g, 5.28 mmol, 48% yield) as a yellow solid. ¹H NMR (400 MHz, CDCl₃) δ 7.50-7.31 (m, 5H), 6.70-6.55 (m, 2H), 5.04 (s, 2H), 3.68 (s, 2H), 2.92-2.82 (m, 2H), 2.54 (t, J=7.0 Hz, 2H), 2.00 (quin, J=6.8 Hz, 2H).

### Step 8: General Procedure of Compound 25-9

To a solution of 25-8 (450 mg, 1.58 mmol, 1 eq) in DCE (10 mL) was added 3-methylbutan-1-amine (206.93 mg, 2.37 mmol, 275.91 μL, 1.5 eq) and allowed to stir for 1 h at 25° C. To this was then added NaBH(OAc)₃ (1.01 g, 4.75 mmol, 3 eq) and allowed to stir for an additional 2 h. To the resulting reaction mixture was then added H₂O (10 mL) then extracted with ethyl acetate (5 mL×3). The combined organic layers were washed with brine (10 mL), dried with Na₂SO₄, then filtered and concentrated under reduced pressure to give 25-9 (560 mg, crude) as a yellow oil.

### Step 9: General Procedure of Compound 25-10

To a solution of 25-9 (560 mg, 1.58 mmol, 1 eq) in THF (15 mL) was added Boc₂O (688 mg, 3.15 mmol, 723 μL, 2 eq) and triethylamine (478 mg, 4.7 mmol, 657 μL, 3 eq) and allowed to stir for 12 h at 25° C. To the resulting reaction mixture was added H₂O (20 mL) then extracted with ethyl acetate (15 mL×3). The combined organic layers were washed with brine (20 mL), dried with Na₂SO₄, then filtered and concentrated under reduced pressure to give a crude. The crude was then purified by column chromatography (SiO₂, petroleum ether/ethyl acetate=3% ethyl acetate) to give 25-10 (580 mg, 1.27 mmol, 80% yield) as a yellow oil.

### Step 10: General Procedure of Compound 25-11

To a solution of 25-10 (580 mg, 1.2 mmol, 1 eq) in THF (10 mL) was added 2.5 M n-BuLi (1.02 mL, 2 eq) dropwise at −78° C. then allowed to stir for 1 h under N₂. To this was then added a solution of I₂ (646 mg, 2.55 mmol, 512 μL, 2 eq) in THF (5 mL) and allowed to stir for an additional 1 h. The resulting suspension was quenched with sat. NH₄Cl(aq) (15 mL) then extracted with ethyl acetate (5 mL×2). The combined organic layers were washed with brine (15 mL), dried over Na₂SO₄, then filtered and concentrated under reduced pressure to give a crude. The crude was then purified by prep-TLC (SiO₂, petroleum ether/ethyl acetate=10/1, Rf=0.4) to give 25-11 (380 mg, 653 μmol, 51% yield) as a white solid. ¹H NMR (400 MHz, CDCl₃) δ 7.50 (d, J=7.3 Hz, 2H), 7.41 (t, J=7.4 Hz, 2H), 7.37-7.31 (m, 1H), 6.47 (s, 1H), 5.14 (d, J=2.3 Hz, 2H), 3.54-2.51 (m, 7H), 2.37-1.85 (m, 3H), 1.53-1.20 (m, 13H), 0.94 (dd, J=0.8, 6.6 Hz, 6H).

### Step 11: General Procedure of Compound 25-12

To a solution of 25-11 (180 mg, 309 μmol, 1 eq) in 1,4-dioxane (9 mL) was added tert-butyl 2-aminoacetate (60 mg, 464 μmol, 1.5 eq) and Cs₂CO₃ (201 mg, 619 μmol, 2 eq) then degassed under vacuum and purged with N₂ for 3 times. To this was then added BrettPhos Pd G3 (28 mg, 30 μmol, 0.1 eq) and again was degassed under vacuum and purged with N₂ for another 3 times. The reaction mixture was then heated to 90° C. and allowed to stir for 16 h. The resulting reaction mixture was then concentrated under reduced pressure to give a crude. The crude was then purified by column chromatography (SiO₂, petroleum ether/ethyl acetate=5/1) to give 25-12 (130 mg, 222 μmol, 71% yield) as a yellow solid. ¹H NMR (400 MHz, CDCl₃) δ 7.54-7.21 (m, 5H), 6.52-6.24 (m, 1H), 5.13-4.93 (m, 2H), 3.49-2.43 (m, 7H), 2.21-2.03 (m, 1H), 1.95-1.86 (m, 2H), 1.77-1.43 (m, 10H), 1.42-1.35 (m, 10H), 0.88-0.82 (m, 6H).

### Step 12: General Procedure of Compound 25-13

To a solution of N-(oxomethylene)sulfamoyl chloride (135 mg, 957 μmol, 83 μL, 2 eq) in CH₂Cl₂ (5 mL) was added prop-2-en-1-ol (55 mg, 957 μmol, 65 μL, 2 eq) and allowed to stir for 0.5 h at 0° C. under N₂. To this was then added a solution of 25-12 (280 mg, 478 μmol, 1 eq) and triethylamine (145 mg, 1.44 mmol, 199 μL, 3 eq) in CH₂Cl₂ (5 mL) and allowed to stir for an additional 0.5 h. The reaction mixture was then allowed to warm to 25° C. and allowed to stir for an additional 1 h. To the resulting reaction mixture was added H₂O (20 mL) then extracted with CH₂Cl₂ (20 mL×3). The combined organic layers were washed with brine (30 mL), dried with Na₂SO₄, then filtered and concentrated under reduced pressure to give 25-13 (400 mg, crude) as a yellow oil.

### Step 13: General Procedure of Compound 25-14

To a mixture of 25-13 (50 mg, 66 μmol, 1 eq) MeOH (1 mL) was added NaOMe (60 mg, 334 μmol, 30% purity, 5 eq), Pd(PPh₃)₄ (7.7 mg, 6.7 μmol, 0.1 eq) then heated to 60° C. and allowed to stir for 1 h under N₂. To the resulting reaction mixture was then added 0.5 M HCl (5 mL) then extracted with ethyl acetate (5 mL×3). The combined organic layers were washed with brine (10 mL), dried with Na₂SO₄, then filtered and concentrated under reduced pressure to give 25-14 (45 mg, crude) as a yellow oil.

### Step 14: General Procedure of Compound 25-15

A mixture of 25-14 (45 mg, 76 μmol, 1 eq) in HCl/ethyl acetate (3 mL) was allowed to stir for 0.5 h at 25° C. The resulting reaction mixture was then concentrated under reduced pressure to give 25-15 (40 mg, 76 μmol, 99% yield, HCl) as a yellow oil.

### Step 15: General Procedure of Example 25

To a solution of 25-15 (30 mg, 61 μmol, 1 eq) in MeOH (5 mL) was added Pd/C (10 mg, 10% purity) then purged with H₂ (15 psi) and allowed to stir for 16 h at 25° C. The reaction mixture was then filtered and concentrated under reduced pressure to give a crude. The crude was then purified by prep-HPLC to give example 25 (3.3 mg, 8.3 μmol, 13% yield). ¹H NMR (400 MHz, CD₃OD) δ 6.59 (s, 1H), 4.24 (s, 2H), 3.25-3.00 (m, 4H), 2.96-2.70 (m, 3H), 2.37-2.20 (m, 1H), 2.07-1.81 (m, 2H), 1.77-1.65 (m, 1H), 1.57 (td, J=6.0, 10.1 Hz, 3H), 0.98 (d, J=6.5 Hz, 6H).

### Step 16: General Procedure of Example 26 and 27

Example 26 and Example 27 were obtained by SFC separation of Example 25 (stereochemistry at chiral center was arbitrarily assigned).

Example 26: LCMS (ESI−): m/z=398.2 (M−H)⁻. ¹H NMR (400 MHz, D₂O) δ 6.67 (s, 1H), 4.34 (s, 2H), 3.37-3.25 (m, 1H), 3.20 (br d, J=14.9 Hz, 1H), 3.16-2.97 (m, 3H), 2.77 (br d, J=4.5 Hz, 2H), 2.28-2.14 (m, 1H), 1.99-1.80 (m, 2H), 1.72-1.43 (m, 4H), 0.89 (d, J=6.6 Hz, 6H).

Example 27: LCMS (ESI−): m/z=398.2 (M−H)⁻. ¹H NMR (400 MHz, D₂O) δ 6.62 (s, 1H), 4.29 (s, 2H), 3.29-3.20 (m, 1H), 3.15 (br d, J=14.4 Hz, 1H), 3.10-2.91 (m, 3H), 2.72 (br d, J=4.1 Hz, 2H), 2.21-2.08 (m, 1H), 1.93-1.75 (m, 2H), 1.64-1.41 (m, 4H), 0.84 (d, J=6.5 Hz, 6H)

### Example 28: 5-(1-fluoro-3-hydroxy-8-(isopentyl(methyl)amino)-6,7,8,9-tetrahydro-5H-benzo[7]annulen-2-yl)-1,2,5-thiadiazolidin-3-one 1,1-dioxide

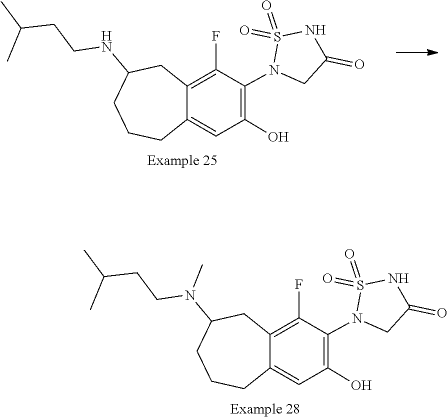 [SMILES: CC(C)CCNC1CCCc2cc(O)c(N3CC(=O)NS3(=O)=O)c(F)c2C1 ; CC(C)CCN(C)C1CCCc2cc(O)c(N3CC(=O)NS3(=O)=O)c(F)c2C1]

To a solution of Example 25 (15 mg, 31 μmol, 1 eq) in MeOH (3 mL) was added formaldehyde (115 mg, 1.5 mmol, 105 μL, 40% purity, 50 eq) and Pd/C (10 mg, 10% purity) then purged with H₂ (15 psi) and allowed to stir for 1 h at 25° C. The reaction mixture was then filtered and concentrated under reduced pressure to give a crude. The crude was then purified by prep-HPLC to give Example 28 (2.0 mg, 4.6 μmol, 15% yield, 95%) as a white solid. LCMS (ESI−): m/z=412.2 (M−H)⁻. ¹H NMR (400 MHz, CD₃OD) δ 6.58 (s, 1H), 4.27 (s, 1H), 3.38 (br s, 1H), 3.28-3.11 (m, 3H), 2.99-2.63 (m, 6H), 2.33-1.95 (m, 3H), 1.79-1.40 (m, 4H), 1.00 (d, J=6.4 Hz, 6H).

### Example 29: 5-(1-fluoro-3-hydroxy-6,7,8,9-tetrahydro-5H-benzo[7]annulen-2-yl)-1,2,5-thiadiazolidin-3-one 1,1-dioxide

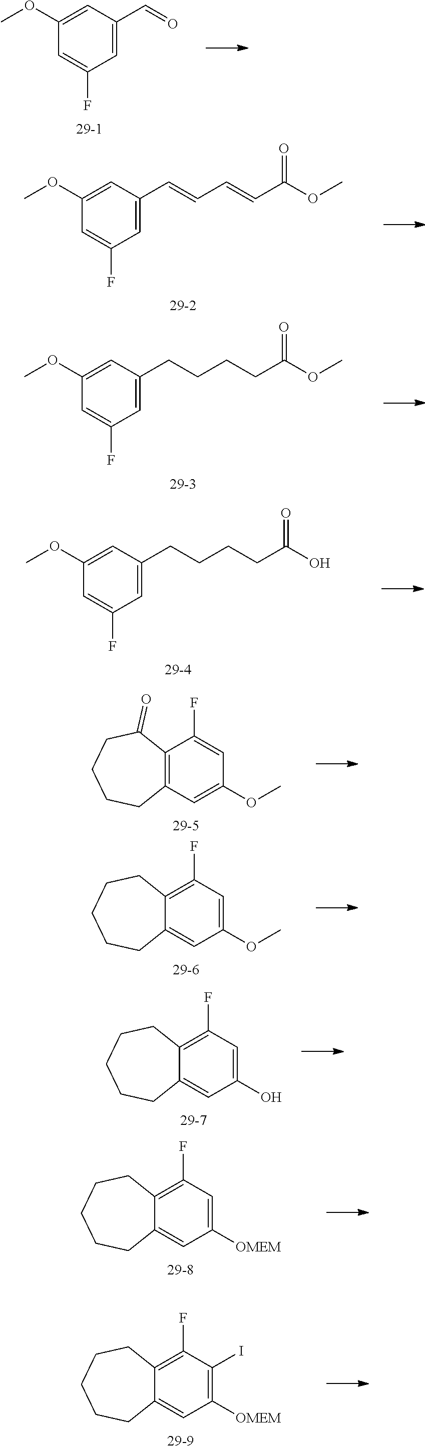

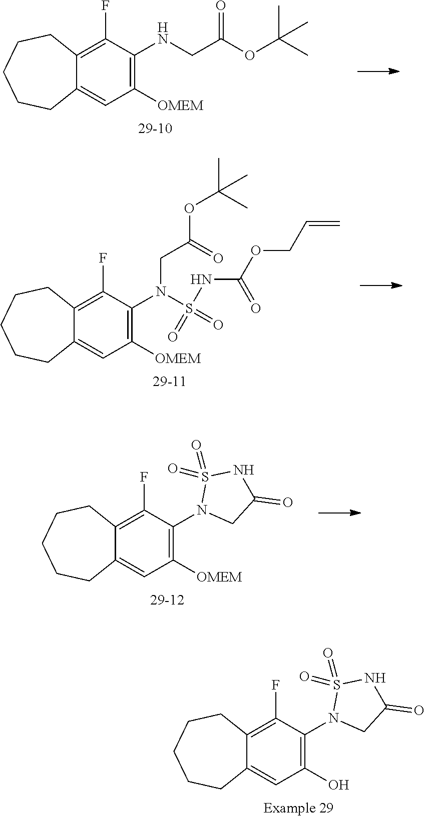

### Step 1: General Procedure of Compound 29-2

To a solution of 29-1 (3 g, 19.4 mmol, 1 eq) in THF (30 mL) was added methyl (E)-4-diethoxyphospho-rylbut-2-enoate (6.9 g, 29 mmol, 1.5 eq) and NaH (1.17 g, 29 mmol, 60% purity, 1.5 eq) then cooled to 0° C. under N₂. This was then heated to 80° C. and allowed to stir for 16 h. The resulting reaction mixture was quenched with sat. NH₄Cl(aq) (50 mL) then extracted with ethyl acetate (50 mL×3). The combined organic layers were washed with brine (50 mL), dried over Na₂SO₄, then filtered and concentrated under reduced pressure to give a crude. The crude was then purified by column chromatography (SiO₂, petroleum ether/ethyl acetate=100/1 to 5/1) to give 29-2 (10.5 g, 44 mmol, 25% yield) as a white solid. ¹H NMR (400 MHz, CDCl₃) δ 7.43 (ddd, J=1.6, 8.7, 15.3 Hz, 1H), 6.86-6.75 (m, 4H), 6.58 (td, J=2.2, 10.4 Hz, 1H), 6.03 (d, J=15.3 Hz, 1H), 3.83 (s, 3H), 3.78 (s, 3H).

### Step 2: General Procedure of Compound 29-3

To a solution of 29-2 (3.5 g, 14.8 mmol, 1 eq) in THF (80 mL) was added Pd/C (1.5 g, 10% purity) then purge with H₂ (15 psi) and allowed to stir for 16 h at 25° C. The reaction mixture was then filtered and concentrated under reduced pressure to give 29-3 (10.8 g, crude) as a colorless oil. ¹H NMR (400 MHz, CDCl₃) δ 6.55-6.39 (m, 3H), 3.79 (s, 3H), 3.68 (s, 3H), 2.59 (t, J=7.1 Hz, 2H), 2.37-2.31 (m, 2H), 1.70-1.61 (m, 4H).

### Step 3: General Procedure of Compound 29-4

To a solution of 29-3 (3.6 g, 14.9 mmol, 1 eq) in H₂O (9 mL), MeOH (18 mL) and THF (18 mL) was added LiOH—H₂O (855 mg, 20.3 mmol, 1.36 eq) at −5° C. The reaction mixture was then allowed to warm to 25° C. and was stirred for 12 h. The resulting reaction mixture was concentrated under reduced pressure, then resuspended in H₂O (50 mL) and extracted with ethyl acetate (50 mL). The aqueous layer was adjusted to pH=2-3 with 2N HCl (aq) then extracted with ethyl acetate (50 mL×3), dried over Na₂SO₄, then filtered and concentrated under reduced pressure to give 29-4 (10.2 g, crude) as an orange solid. ¹H NMR (400 MHz, CDCl₃) δ 6.59-6.39 (m, 3H), 3.79 (s, 3H), 2.60 (br t, J=6.8 Hz, 2H), 2.44-2.33 (m, 2H), 1.68 (td, J=3.5, 7.0 Hz, 4H).

### Step 4: General Procedure of Compound 29-5

To a solution of 29-4 (3.4 g, 15.03 mmol, 1 eq) in chlorobenzene (35 mL) was added PPA (3.4 g) then heated to 80° C. and allowed to stir for 12 h. The resulting reaction mixture was concentrated under reduced pressure, then resuspended in ethyl acetate (70 mL) and washed with 1N NaOH (aq) (30 mL) then brine (30 mL). The combined organic layers were dried over Na₂SO₄ then filtered and concentrated under reduced pressure to give a crude. The crude was then purified by column chromatography (SiO₂, petroleum ether/ethyl acetate=100/1 to 5/1) to give 29-5 (5.5 g, 26.4 mmol, 58% yield) as a yellow oil.

¹H NMR (400 MHz, CDCl₃) δ 6.62-6.40 (m, 2H), 3.85-3.74 (m, 3H), 2.81 (t, J=6.3 Hz, 1H), 2.73 (br t, J=6.2 Hz, 1H), 2.68-2.59 (m, 2H), 1.89-1.75 (m, 4H).

### Step 5: General Procedure of Compound 29-6

To a solution of 29-5 (1.1 g, 5.28 mmol, 1 eq) in TFA (11 mL) was added triethylsilane (3.07 g, 26.4 mmol, 4.2 mL, 5 eq) dropwise under N₂. The reaction mixture was then heated to 60° C. and allowed to stir for 12 h. To the resulting reaction mixture was added H₂O (25 mL) then extracted with ethyl acetate (25 mL×3). The combined organic layers were washed with brine (30 mL), dried with Na₂SO₄, then filtered and concentrated under reduced pressure to give a crude. The crude was then purified by column chromatography (SiO₂, petroleum ether/ethyl acetate=1/0 to 99/1) to give 29-6 (2.7 g, crude) as brown oil. ¹H NMR (400 MHz, CDCl₃) δ 6.58-6.33 (m, 2H), 3.77 (s, 3H), 2.85-2.68 (m, 4H), 1.89-1.76 (m, 2H), 1.70-1.57 (m, 4H).

### Step 6: General Procedure of Compound 29-7

To a solution of 29-6 (0.9 g, 4.63 mmol, 1 eq) in CH₂Cl₂ (30 mL) was added BBr₃ (5.8 g, 23.1 mmol, 2.23 mL, 5 eq) dropwise at 0° C. and allowed to stir for 30 mins under N₂. The resulting reaction mixture was poured into 1M HCl (50 mL) and stirred for 5 min, then extracted with CH₂Cl₂ (50 mL×3). The combined organic layers were washed with brine (30 mL), dried over Na₂SO₄, then filtered and concentrated under reduced pressure to give a crude. The crude was then purified by column chromatography (SiO₂, petroleum ether/ethyl acetate=10/1 to 9/1) to give 29-7 (2.4 g, 13.32 mmol, 95% yield) as colorless oil. ¹H NMR (400 MHz, CDCl₃) δ 6.48-6.30 (m, 2H), 4.63 (s, 1H), 2.85-2.62 (m, 4H), 1.91-1.77 (m, 2H), 1.61 (tdd, J=5.6, 10.9, 16.6 Hz, 4H).

### Step 7: General Procedure of Compound 29-8

To a solution of 29-8 (0.44 g, 2.4 mmol, 1 eq) in THF (5 mL) was added 1-(chloromethoxy)-2-methoxy-ethane (456 mg, 3.6 mmol, 418 μL, 1.5 eq) and NaH (195 mg, 4.8 mmol, 60% purity, 2 eq) at 0° C. The reaction mixture was then allowed to warm to 25° C. and stirred for 12 h. The resulting reaction mixture was quenched with sat. NH₄Cl(aq) (20 mL) then extracted with ethyl acetate (15 mL×3). The combined organic layers were washed with brine (30 mL), dried over Na₂SO₄, then filtered and concentrated under reduced pressure to give a crude. The crude was then purified by prep-TLC (SiO₂, petroleum ether/ethyl acetate=5:1, Rf=0.52) to give 29-8 (2.35 g, 8.7 mmol, 71% yield) as brown oil. ¹H NMR (400 MHz, CDCl₃) δ 6.68-6.57 (m, 2H), 5.22 (s, 2H), 3.82 (dd, J=3.9, 5.4 Hz, 2H), 3.57 (dd, J=3.8, 5.5 Hz, 2H), 3.39 (s, 3H), 2.76 (br dd, J=3.4, 7.2 Hz, 4H), 1.87-1.78 (m, 2H), 1.61 (dt, J=5.1, 10.1 Hz, 4H).

### Step 8: General Procedure of Compound 29-9

To a solution of 2,2,6,6-tetramethylpiperidine (494 mg, 3.5 mmol, 594 μL, 2 eq) in THF (5 mL) was added 2.5M n-BuLi (1.40 mL, 2 eq) dropwise at 0° C. and allowed to stir for 0.5 h under N₂. The reaction mixture was then cooled to −78° C. and added a solution of 29-8 (470 mg, 1.75 mmol, 1 eq) in THF (5 mL) and N,N,N,N-tetramethylethane-1,2-diamine (305 mg, 2.63 mmol, 396 μL, 1.5 eq) then allowed to stir for an additional 2 h. To the reaction mixture was then added a solution of I₂ (1.11 g, 4.38 mmol, 882 μL, 2.5 eq) in THF (5 mL) and stirred for an additional 10 min. The resulting suspension was quenched with a mixture of sat. NH₄Cl(aq) and 1M Na₂S₂O₃(aq) (1:1, 20 mL) and allowed to stir for 5 minutes at 25° C. The resulting reaction mixture was then extracted with ethyl acetate (15 mL×3). The combined organic layers were washed with water (20 mL) and brine (20 mL), dried over Na₂SO₄, then filtered and concentrated under reduced pressure to give a crude. The crude was then purified by prep-TLC (SiO₂, petroleum ether/ethyl acetate=10:1, Rf=0.45) to give 29-9 (3 g, 7.61 mmol, 86% yield) as a brown oil. ¹H NMR (400 MHz, CDCl₃) δ 6.73 (s, 1H), 5.32 (s, 2H), 3.94-3.81 (m, 2H), 3.65-3.53 (m, 2H), 3.39 (s, 3H), 2.89-2.69 (m, 4H), 1.88-1.77 (m, 2H), 1.67-1.57 (m, 4H).

### Step 9: General Procedure of Compound 29-10

To a solution of 29-9 (1.5 g, 3.80 mmol, 1 eq) in 1,4-dioxane (15 mL) was added tert-butyl 2-aminoacetate (748 mg, 5.7 mmol, 1.5 eq) and Cs₂CO₃ (2.48 g, 7.61 mmol, 2 eq) then degassed under vacuum and purged with N₂ for 3 times. To this was then added dicyclohexyl-[3,6-dimethoxy-2-(2,4,6-triisopropyl-phenyl)phenyl]phosphane (204 mg, 380 μmol, 0.1 eq) and [2-(2-aminophenyl)phenyl]-methylsulfonyl-oxy-palladium, dicyclohexyl-[3,6-dimethoxy-2-(2,4,6-triisopropylphenyl)phenyl]-phosphane (344 mg, 380 μmol, 0.1 eq) and again degassed under vacuum and purged with N₂ for 3 times. The reaction mixture was then heated to 90° C. and allowed to stir for 16 h. To the resulting reaction mixture was added H₂O (30 mL) then extracted with ethyl acetate (25 mL×3). The combined organic layers were washed with brine (30 mL), dried with Na₂SO₄, then filtered and concentrated under reduced pressure to give a crude. The crude was then purified by column chromatography (SiO₂, petroleum ether/ethyl acetate=100/1 to 5/1) to give 29-10 (2.1 g, 5.28 mmol, 69% yield) as a brown oil. ¹H NMR (400 MHz, CDCl₃) δ 6.69 (s, 1H), 5.26 (s, 2H), 3.95 (d, J=1.9 Hz, 2H), 3.86 (dd, J=3.8, 5.5 Hz, 2H), 3.61-3.55 (m, 2H), 3.39 (s, 3H), 2.78-2.65 (m, 4H), 1.83-1.74 (m, 2H), 1.64-1.55 (m, 4H), 1.45 (s, 9H).

### Step 10: General Procedure of Compound 29-11

To a solution of N-(oxomethylene)sulfamoyl chloride (1.44 g, 10.19 mmol, 884 μL, 4.5 eq) in CH₂Cl₂ (15 mL) was added prop-2-en-1-ol (591 mg, 10.19 mmol, 692 μL, 4.5 eq) and allowed to stir for 0.5 h at 0° C. under N₂. To this was then added a solution of 29-10 (0.9 g, 2.26 mmol, 1 eq) and N,N-diethylethan-amine (458 mg, 4.53 mmol, 630 μL, 2 eq) in CH₂Cl₂ (10 mL) and stirred for an additional 0.5 h. To the resulting reaction mixture was added H₂O (30 mL) then extracted with CH₂Cl₂ (30 mL×3). The combined organic layers were washed with brine (30 mL), dried with Na₂SO₄, then filtered and concentrated under reduced pressure to give 29-11 (1.5 g, crude) as a brown oil. ¹H NMR (400 MHz, CDCl₃) δ 6.80 (s, 1H), 6.04-5.82 (m, 1H), 5.38 (dd, J=1.4, 17.1 Hz, 1H), 5.30-5.25 (m, 2H), 4.68 (d, J=5.8 Hz, 2H), 4.23 (d, J=17.8 Hz, 1H), 3.86 (td, J=3.7, 5.7 Hz, 2H), 3.55 (dt, J=3.5, 5.7 Hz, 2H), 3.35 (s, 3H), 2.84-2.70 (m, 4H), 1.86-1.78 (m, 2H), 1.68-1.55 (m, 4H), 1.44 (s, 9H).

### Step 11: General Procedure of Compound 29-12

To a solution of 29-11 (0.5 g, 891 μmol, 1 eq) in MeOH (5 mL) was added NaOMe (481 mg, 2.68 mmol, 30% purity, 3 eq) and Pd(PPh₃)₄ (30 mg, 26 μmol, 0.03 eq) then heated to 60° C. and allowed to stir for 2 h under N₂. The resulting suspension was quenched by addition of 1M HCl (30 mL) then extracted with ethyl acetate (20 mL×3). The combined organic layers were washed with brine (20 mL), dried over Na₂SO₄, then filtered and concentrated under reduced pressure to give 29-12 (1.1 g, crude) as a yellow oil. ¹H NMR (400 MHz, CDCl₃) δ 6.82 (s, 1H), 5.31 (s, 2H), 4.42 (s, 2H), 3.86 (dd, J=3.6, 5.4 Hz, 2H), 3.62-3.53 (m, 2H), 3.47-3.32 (m, 3H), 2.84-2.72 (m, 4H), 1.88-1.78 (m, 2H), 1.64 (br dd, J=5.4, 11.0 Hz, 4H).

### Step 12: General Procedure of Compound Example 29

A solution of 29-12 (500 mg, 1.24 mmol, 1 eq) in 4M HCl/dioxane (20 mL, 64 eq) was allowed to stir for 30 min at 25° C. The reaction mixture was then concentrated under reduced pressure to give a crude. The crude was then purified by prep-HPLC to give Example 29 (212 mg, 653 μmol, 26% yield). LCMS (ESI−): m/z=313.1 (M−H)⁻. ¹H NMR (400 MHz, DMSO-d₆) δ 6.54 (s, 1H), 4.38 (s, 2H), 2.76-2.58 (m, 4H), 1.76 (br d, J=4.1 Hz, 2H), 1.61-1.46 (m, 4H).

### Example 30: 5-(6-fluoro-8-hydroxy-2,3,4,5-tetrahydro-1H-benzo[d]azepin-7-yl)-1,2,5-thiadiazolidin-3-one 1,1-dioxide

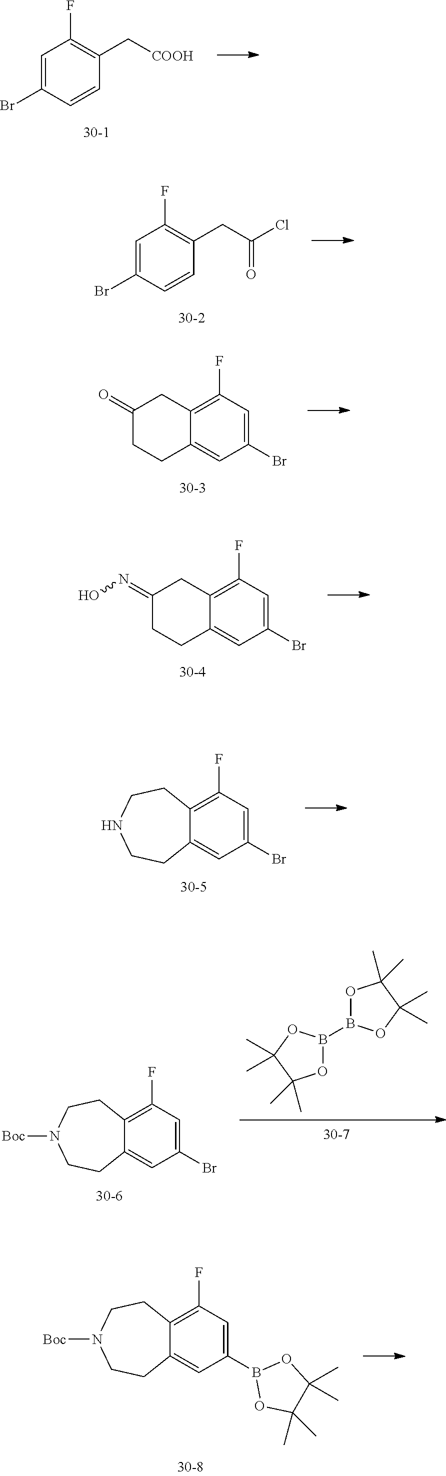

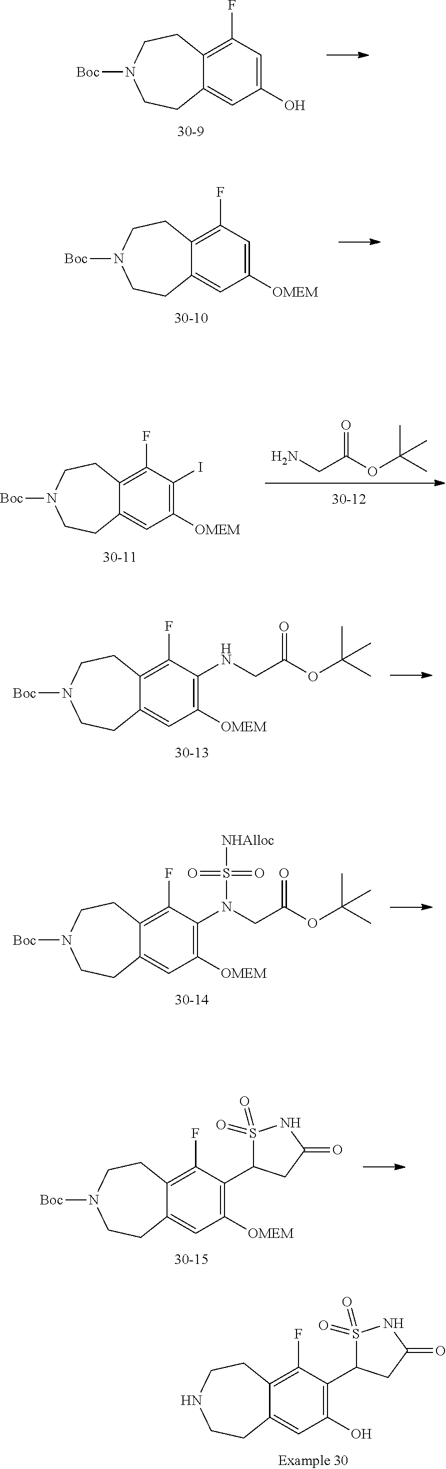

### Step 1: General Procedure of Compound 30-2

To a solution of 30-1 (200 g, 858 mmol, 1 eq) in CH₂Cl₂ (1 L) was added (COCl)₂ (163.4 g, 1.29 mol, 112 mL, 1.5 eq) and DMF (12.5 g, 171.6 mmol, 13.2 mL, 0.2 eq) and allowed to stir for 1 h at 0° C. under N₂. The reaction mixture was then concentrated under reduced pressure to give 30-2 (220 g, crude) as a brown oil. LCMS (ESI+): m/z=245.1/247.1 (M−OCl+MeOH)⁺.

### Step 2: General Procedure of Compound 30-3

To a solution of AlCl₃ (349.9 g, 2.62 mol, 143 mL, 3 eq) in CH₂Cl₂ (1.5 L) was added 30-2 (220 g, 874 mmol, 1 eq) in CH₂Cl₂ (500 mL) at 0° C. The reaction mixture was then allowed to stir for 1 h followed by addition of ethylene, then allowed to stir for an additional 2 h. To this was then added H₂O (1 L) then extracted with CH₂Cl₂ (1.2 L×2). The combined organic layers were washed with brine (500 mL), dried over Na₂SO₄, then filtered and concentrated under reduced pressure to give a crude. The crude was then purified by column chromatography (SiO₂, petroleum ether/ethyl acetate=1/0 to 0/1) to give 30-3 (130 g, 534.82 mmol, 61% yield). ¹H NMR (CDCl₃) δ 7.21 (s, 1H), 7.18-7.12 (m, 1H), 3.51 (s, 2H), 3.08 (t, J=6.8 Hz, 2H), 2.63-2.55 (m, 2H).

### Step 3: General Procedure of Compound 30-4

To a solution of 30-3 (130 g, 534 mmol, 1 eq) in MeOH (1.3 L) was added NH₂OH—HCl (55.7 g, 802 mmol, 1.5 eq) and NaOAc (87.7 g, 1.07 mol, 2 eq) and allowed to stir for 1.5 h at 25° C. The resulting reaction mixture was concentrated under reduced pressure to remove MeOH. To this was added H₂O (300 mL) then extracted with ethyl acetate (150 mL×3). The combined organic layers were washed with brine (300 mL), dried over Na₂SO₄, then filtered and concentrated under reduced pressure to give a crude. The crude was triturated with petroleum ether to give 30-4 (100 g, 387 mmol, 72% yield) as a pale solid. ¹H NMR (CDCl₃) δ 9.02-8.39 (m, 1H), 7.16 (s, 1H), 7.14-7.09 (m, 2H), 3.74 (s, 2H), 3.55-3.48 (m, 1H), 2.95-2.84 (m, 3H), 2.79-2.71 (m, 1H), 2.60-2.52 (m, 2H).

### Step 4: General Procedure of Compound 30-5

To a solution of 30-4 (6 g, 23.25 mmol, 1 eq) in CH₂Cl₂ (60 mL) was added 1M DIBALH (116 mL, 5 eq) dropwise at 0° C. under N₂. The reaction mixture was then allowed to stir for 2 h at 25° C. The reaction mixture was then cooled to 0° C. and added NaF (9.7 g, 232 mmol, 9.7 mL, 10 eq) in H₂O (20 mL). The reaction mixture was then allowed to warm to 25° C. and stirred for an additional 2 h. The resulting suspension was quenched with a mixture of sat. NH₄Cl(aq) and 1M Na₂S₂O₃(aq) (1:1, 100 mL) and allowed to stir for 5 minutes at 25° C. The resulting reaction mixture was then extracted with ethyl acetate (100 mL×2). The combined organic layers were washed with water (100 mL) and brine (100 mL), dried over Na₂SO₄, then filtered and concentrated under reduced pressure to give a crude. The crude was then purified by HPLC to give 30-5 (1 g, 4.10 mmol, 17% yield) as a white solid. ¹H NMR (CD₃OD) δ 7.31-7.27 (m, 2H), 3.33-3.29 (m, 4H), 3.22-3.17 (m, 4H).

### Step 5: General Procedure of Compound 30-6

To a solution of 30-5 (0.5 g, 2.05 mmol, 1 eq) in CH₂Cl₂ (5 mL) was added Boc₂O (894 mg, 4.1 mmol, 941, 2 eq) and DMAP (25.02 mg, 204.83 μmol, 0.1 eq) then allowed to stir for 12 h at 25° C. The resulting suspension was quenched with a mixture of sat. NH₄Cl(aq) and 1M Na₂S₂O₃(aq) (1:1, 10 mL) and allowed to stir for 5 minutes at 25° C. The resulting reaction mixture was then extracted with ethyl acetate (10 mL×2). The combined organic layers were washed with water (10 mL) and brine (10 mL), dried over Na₂SO₄, then filtered and concentrated under reduced pressure to give a crude. The crude was then purified by column chromatography (SiO₂, petroleum ether/ethyl acetate=1/0 to 0/1) to give 30-6 (0.7 g, 2.03 mmol, 99% yield) as a yellow oil. ¹H NMR (CDCl₃) δ 7.12-7.07 (m, 2H), 3.59-3.50 (m, 4H), 2.94-2.86 (m, 4H), 1.48 (s, 9H).

### Step 6: General Procedure of Compound 30-8

To a solution of 30-6 (0.7 g, 2.03 mmol, 1 eq) in 1,4-dioxane (10 mL) was added 30-7 (1.03 g, 4.07 mmol, 2 eq), Pd(dppf)Cl₂ (148 mg, 203 μmol, 0.1 eq) and KOAc (798 mg, 8.1 mmol, 4 eq) then heated to 90° C. for 2 h under N₂. The resulting suspension was quenched with a mixture of sat. NH₄Cl (aq) and 1M Na₂S₂O₃(aq) (1:1, 10 mL) and allowed to stir for 5 minutes at 25° C. The resulting reaction mixture was then extracted with ethyl acetate (10 mL×2). The combined organic layers were washed with water (10 mL) and brine (10 mL), dried over Na₂SO₄, then filtered and concentrated under reduced pressure to give a crude. The crude was then purified by column chromatography (SiO₂, petroleum ether/ethyl acetate=1/0 to 0/1) to give 30-8 (1.3 g, crude) as yellow solid. ¹H NMR (CDCl₃) δ 7.34-7.30 (m, 2H), 3.57-3.51 (m, 4H), 2.99-2.90 (m, 4H), 1.33 (s, 9H), 1.26 (s, 12H).

### Step 7: General Procedure of Compound 30-9

To a solution of 30-8 (1.2 g, 3.07 mmol, 1 eq) in acetone (20 mL) was added oxone (3.77 g, 6.13 mmol, 2 eq) in H₂O (20 mL) then was allowed to stir for 0.5 h at 0° C. To this was added H₂O (50 mL) then extracted with ethyl acetate (50 mL×3). The combined organic layers were washed with brine (50 mL), dried over Na₂SO₄, then filtered and concentrated under reduced pressure to give a crude. The crude was then purified by column chromatography (SiO₂, petroleum ether/ethyl acetate=1/0 to 0/1) to give 30-9 as colorless oil. ¹H NMR (CDCl₃) δ 6.49-6.43 (m, 2H), 3.58-3.50 (m, 4H), 2.89-2.82 (m, 4H), 1.49 (s, 9H).

### Step 8: General Procedure of Compound 30-10

To a solution of 30-9 (0.7 g, 2.49 mmol, 1 eq) in THF (10 mL) was added NaH (199 mg, 4.9 mmol, 60% purity, 2 eq) then was allowed to stir for 0.5 h at 0° C. under N₂. To the reaction mixture was then added MEMCl (464 mg, 3.73 mmol, 426 μL, 1.5 eq) then allowed to warm to 25° C. and stirred for an additional 12 hr. The resulting suspension was quenched with a mixture of sat. NH₄Cl(aq) and 1M Na₂S₂O₃(aq) (1:1, 20 mL) and allowed to stir for 5 minutes at 25° C. The resulting reaction mixture was then extracted with ethyl acetate (10 mL×2). The combined organic layers were washed with water (20 mL) and brine (30 mL), dried over Na₂SO₄, then filtered and concentrated under reduced pressure to give a crude. The crude was then purified by column chromatography (SiO₂, petroleum ether/ethyl acetate=1/0 to 0/1) to give 30-10 (0.7 g, 1.89 mmol, 76% yield) as yellow solid. ¹H NMR (CDCl₃) δ 6.69-6.61 (m, 2H), 5.23 (s, 2H), 3.83-3.80 (m, 2H), 3.59-3.49 (m, 6H), 3.39 (s, 3H), 2.87 (br d, J=4.8 Hz, 4H), 1.49 (s, 9H).

### Step 9: General Procedure of Compound 30-11

To a solution of 2,2,6,6-Tetramethylpiperidine (497 mg, 3.5 mmol, 597 μL, 2 eq) in THF (6 mL) was added 2.5M n-BuLi (1.41 mL, 2 eq) dropwise at 0° C. then was allowed to stir for 0.5 h under N₂. The reaction mixture was then cooled to −78° C. then added 30-10 (0.65 g, 1.76 mmol, 1 eq) in THF (3 mL) followed by tetramethylethylenediamine (306 mg, 2.64 mmol, 398 μL, 1.5 eq) and allowed to stir for an additional 2 h. To this was then added I₂ (1.12 g, 4.4 mmol, 886 μL, 2.5 eq) in THF (1.5 mL). The reaction mixture was then allowed to warm to 25° C. and allowed to stir for an additional 1 h. The resulting suspension was quenched with a mixture of sat. NH₄Cl(aq) and 1M Na₂S₂O₃(aq) (1:1, 10 mL) and allowed to stir for 5 minutes at 25° C. The resulting reaction mixture was then extracted with ethyl acetate (10 mL×2). The combined organic layers were washed with water (20 mL) and brine (20 mL), dried over Na₂SO₄, then filtered and concentrated under reduced pressure to give a crude. The crude was then purified by column chromatography to give 30-11 (0.71 g, 1.43 mmol, 81% yield) as a yellow solid. ¹H NMR (CDCl₃) δ 6.73 (s, 1H), 5.32 (s, 2H), 3.90-3.84 (m, 2H), 3.59-3.49 (m, 6H), 3.38 (s, 3H), 2.96-2.85 (m, 4H), 1.48 (s, 9H).

### Step 10: General Procedure of Compound 30-13

To a solution of 30-11 (0.65 g, 1.31 mmol, 1 eq) in 1,4-dioxane (7 mL) was added 30-12 (258 mg, 1.97 mmol, 1.5 eq), Cs₂CO₃ (1.28 g, 3.94 mmol, 3 eq), XPhos (125.12 mg, 262.46 μmol, 0.2 eq) and BrettPhos Pd G₃ (118 mg, 131 μmol, 0.1 eq) then heated to 90° C. for 48 h under N₂. The resulting suspension was quenched with a mixture of sat. NH₄Cl(aq) and 1M Na₂S₂O₃(aq) (1:1, 15 mL) and allowed to stir for 5 minutes at 25° C. The resulting reaction mixture was then extracted with ethyl acetate (10 mL×2). The combined organic layers were washed with water (10 mL) and brine (10 mL), dried over Na₂SO₄, then filtered and concentrated under reduced pressure to give a crude. The crude was then purified by column chromatography (SiO₂, petroleum ether/ethyl acetate=1/0 to 0/1) to give 30-13 (0.5 g, 1.0 mmol, 76% yield) as a yellow oil. ¹H NMR (CDCl₃) δ 6.70 (br s, 1H), 5.26 (br s, 2H), 3.95 (br s, 2H), 3.91-3.80 (m, 2H), 3.64-3.48 (m, 6H), 3.47-3.33 (m, 3H), 3.07-2.64 (m, 4H), 1.67-1.41 (m, 18H).

### Step 11: General Procedure of Compound 30-14

To a solution of N-(oxomethylene)sulfamoyl chloride (191 mg, 1.35 mmol, 117 μL, 1.5 eq) in CH₂Cl₂ (5 mL) was added prop-2-en-1-ol (157 mg, 2.7 mmol, 184 μL, 3 eq) and stirred for 0.5 h at 0° C. The reaction mixture was then allowed to warm to at 25° C. then was added 30-13 (0.45 g, 902 μmol, 1 eq) and diisopropylethylamine (116 mg, 902 μmol, 157 μL, 1 eq) in CH₂Cl₂ (2 mL) and allowed to stir for an additional 1 h. To the reaction mixture was then added H₂O (5 mL) then extracted with CH₂Cl₂ (5 mL×2). The combined organic layers were washed with brine (10 mL), dried over Na₂SO₄, then filtered and concentrated under reduced pressure to give 30-14 (0.5 g, crude) as a yellow oil.

### Step 12: General Procedure of Compound 30-15

To a solution of 30-14 (0.3 g, 453 μmol, 1 eq) in MeOH (3 mL) was added NaOMe (408 mg, 2.27 mmol, 30% purity, 5 eq) and Pd(PPh₃)₄ (52 mg, 45 μmol, 0.1 eq) then heated to 60° C. for 1 h under N₂. To the reaction mixture was then added H₂O (5 mL) then extracted with CH₂Cl₂ (5 mL×2). The combined organic layers were washed with brine (10 mL), dried over Na₂SO₄, then filtered and concentrated under reduced pressure to give 30-15. ¹H NMR (CD₃OD) δ 6.90 (s, 1H), 5.29 (s, 2H), 4.24 (s, 2H), 3.88-3.81 (m, 2H), 3.62-3.50 (m, 6H), 3.34-3.32 (m, 3H), 3.00-2.87 (m, 4H), 1.46 (s, 9H).

### Step 13: General Procedure of Compound Example 30

A solution of 30-15 (0.15 g, 297 μmol, 1 eq) in 4M HCl/EtOAc (5 mL) was allowed to stir for 1 h at 25° C. The resulting reaction mixture was filtered and concentrated under reduced pressure to give a crude. The crude was the purified by HPLC to give Example 30 (0.1 g). LCMS (ESI−): m/z=314.0 (M−H)⁻. ¹H NMR (D₂O) δ 6.64 (s, 1H), 4.31 (s, 2H), 3.26 (br t, J=9.4 Hz, 4H), 3.09-3.02 (m, 4H).

### Example 31: 5-(6-fluoro-8-hydroxy-3-(4-methylpentyl)-2,3,4,5-tetrahydro-1H-benzo[d]azepin-7-yl)-1,2,5-thiadiazolidin-3-one 1,1-dioxide

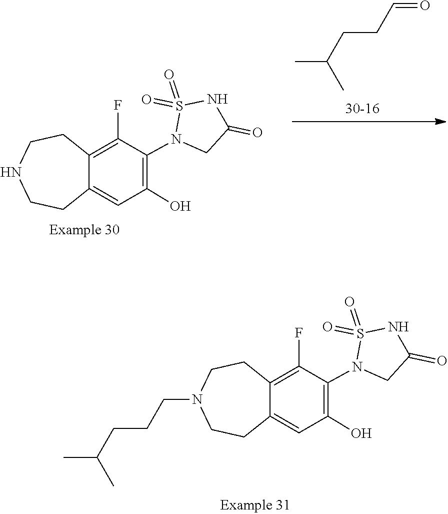

To a solution of Example 30 (0.15 g, 475 μmol, 1 eq) in acetonitrile (5 mL) was added 30-16 (71 mg, 713 μmol, 1.5 eq) and diisopropylethylamine (61 mg, 475 μmol, 82 μL, 1 eq) and allowed to stir for 1 h at 25° C. To this was then added NaBH₃CN (119 mg, 1.9 mmol, 4 eq) and stirred for an additional 12 h.

The resulting reaction mixture was filtered and concentrated under reduced pressure to give a crude. The crude was purified by HPLC to give Example 31 (22 mg). LCMS (ESI+): m/z=400.3 (M+H)⁺. ¹H NMR (D₂O) δ 6.68 (s, 1H), 4.33 (s, 2H), 3.37 (br s, 4H), 3.19-3.04 (m, 6H), 1.78-1.66 (m, 2H), 1.61-1.50 (m, 1H), 1.27-1.15 (m, 2H), 0.85 (d, J=6.6 Hz, 6H).

### Example 33: 5-(6-fluoro-8-hydroxy-3-(4-methylpentyl)-2,3,4,5-tetrahydro-1H-benzo[d]azepin-7-yl)-1,2,5-thiadiazolidin-3-one 1,1-dioxide

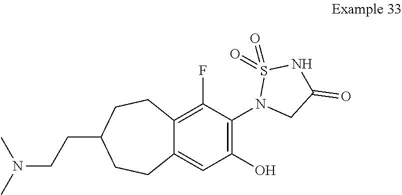 [SMILES: CN(C)CCC1CCc2cc(O)c(N3CC(=O)NS3(=O)=O)c(F)c2CC1]

Example 33 was synthesized as described in any of the examples above. LCMS (ESI−): m/z=384.1 (M−H)⁻. ¹H NMR (400 MHz, DMSO-d₆) δ 8.99 (s, 1H), 6.59 (s, 1H), 3.91 (s, 2H), 2.94-2.97 (m, 2H), 2.64-2.66 (m, 6H), 2.32 (m, 1H), 1.86-1.88 (m, 2H), 1.50-1.52 (m, 3H), 1.22-1.23 (m, 3H), 1.01 (m, 2H).

### Example A: Enzymatic Assay Used to Determine Potency of PTPN2 Inhibitors

Compound activity was determined in an in vitro enzymatic assay using untagged, full-length human PTPN2 (TC45) (1-387) protein. PTPN2 was produced in E. coli as a GST-TEV fusion and the GST was removed by TEV digestion, followed by additional purification to yield full-length PTPN2 (SEQ ID 1). PTPN2 enzyme was diluted in assay buffer (50 mM HEPES pH7.5, 0.2 mM EDTA, 1 mM DTT, 0.02% Brij-35, 0.02% BSA) to a final concentration of 0.5 nM and added to black 384-well non-binding plates (Greiner, 781900). Compounds were subsequently added using a Tecan D300e dispenser. Following a 10 min incubation at room temperature, DiFMUP substrate (ThermoFisher, D22065) was added to a final concentration of 100 μM. Plates were transferred to a SpectraMax plate reader (Molecular Devices) and fluorescence intensity was measured (ex 358, em 455) after a 30 min incubation at room temperature. Each plate included a 100% inhibition control (no enzyme) and a 0% inhibition control (DMSO) from which % inhibition for test compounds was calculated. A four-parameter curve fit was used to determine IC₅₀ values from % inhibition data.

### Example B: B16F10 Cellular Growth Inhibition Assay

Compound activity was determined using an interferon gamma (IFNγ)-induced cellular growth inhibition assay with the murine B16F10 melanoma cell line on an Agilent xCELLigence Real-Time Cell Analysis platform (RTCA). RTCA E-Plate View 96 plates (Agilent, 300601010) were pre-equilibrated with 50 μL of assay media (DMEM+10% FBS, Gibco 10566-024, Gibco 10082-147) at 37° C. in a humidified incubator before taking an initial measurement of impedance (sweep). B16F10 cells cultured in assay media were dissociated with TrypLE Express (Gibco 12605-010) for five minutes at 37° C., diluted in 3 volumes of assay buffer, centrifuged for 5 minutes at 500×g at room temperature before diluting cells to 7,700 cells/mL in assay media, plating 130 μL/well (1,000 cells/well) in the inner 60 wells of the assay plate, and adding 150 μL of assay media to the outer wells of the plate. Cells were incubated at room temperature for 20 min to allow cells to settle before placing them in the xCELLigence reader and incubating overnight at 37° C., sweeping wells every 15 minutes. After 24 hours, well readings were paused, plates were removed from the incubator and compounds were added using a Tecan D300e dispenser. All wells were normalized to a final concentration of 0.5% DMSO. Following a 30 min incubation at 37° C., recombinant mouse IFNγ (R&D Systems™ 485MI100) was diluted to 10 ng/mL in assay media and 20 μL was added to assay wells (1 ng/mL final concentration). Assay plates were placed in the xCELLigence reader and swept every 15 minutes. After 48 hours, well readings were normalized to the time point immediately preceding compound addition and the area under the growth curve (AUC) was calculated by the RTCA software and exported. A four-parameter curve fit was used to determine compound IC₅₀ values using % inhibition for each compound concentration calculated using the DMSO vehicle with IFNγ treatment as baseline (0% inhibition) and a positive control PTPN2 inhibitor with IFNγ treatment as 100% inhibition.

The data from Example A and Example B is shown in table 3.

TABLE 3

| | Full Length PTPN2 DiFMUP | B16 IFNγ Mediated Growth |
|---|---|---|
| Ex | Assay: AVG IC₅₀ (μM) | Inhibition Assay: AVG IC₅₀ (μM) |
| 1 | 0.00507 | |
| 2 | 0.176 | |
| 3 | 0.0165 | 5.14 |
| 4 | 0.0149 | 2.89 |
| 5 | 0.0175 | 2.49 |
| 6 | 0.00567 | 1.63 |
| 7 | 0.444 | >100 |
| 8 | 0.0473 | 1.59 |
| 9 | 0.0216 | 4.15 |
| 10 | 0.005 | 6.63 |
| 11 | 0.149 | >100 |
| 12 | 0.0198 | 4.06 |
| 13 | 0.0853 | >100 |
| 14 | 0.0136 | 15.2 |
| 15 | 0.0795 | 16.8 |
| 16 | 0.0107 | 6.66 |
| 17 | 0.093 | 13.5 |
| 18 | 0.879 | >100 |
| 19 | 0.0124 | 2.69 |
| 20 | 0.0127 | 4.13 |
| 21 | 0.374 | 54 |
| 22 | 0.0245 | |
| 23 | 0.002 | 2.16 |
| 24 | 0.075 | |
| 25 | 0.0259 | 7.08 |
| 26 | 0.0198 | 5.02 |
| 27 | 0.215 | >71.1 |
| 28 | 0.0399 | >61.2 |
| 29 | 0.109 | 13.6 |
| 30 | 0.00228 | 0.996 |
| 31 | 0.00106 | 1.73 |
| 32 | 0.0469 | 14.9 |
| 33 | 0.0211 | |

What is claimed is:

1. A compound of Formula (I), or a pharmaceutically acceptable salt, solvate, or stereoisomer thereof: 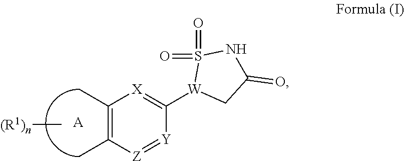
wherein:
Ring A is a 7- to 15-membered cycloalkyl or a 7- to 15-membered heterocycloalkyl comprising 1 to 4 heteroatoms selected from O, S, and N;
each R¹ is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl; wherein each alkyl, cycloalkyl, heterocycloalkyl, aryl, and heteroaryl is independently and optionally substituted with one or more R^{1a};
or two R¹ on the same atom are taken together to form an oxo;
each R^{1a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl; wherein each alkyl, cycloalkyl, heterocycloalkyl, aryl, and heteroaryl is independently and optionally substituted with one or more R;
or two R^{1a} on the same atom are taken together to form an oxo;
n is 0-6;
X is CR^{X} or N;
R^{X} is hydrogen, deuterium, halogen, —CN, —NO₂, —OH, —OR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl;
Y is CR^{Y} or N;
R^{Y} is hydrogen, deuterium, halogen, —CN, —NO₂, —OH, —OR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl;
Z is CR^{Z} or N;
R^{Z} is hydrogen, deuterium, halogen, —CN, —NO₂, —OH, —OR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl;
W is CR^{W} or N;
R^{W} is hydrogen, deuterium, halogen, —CN, —NO₂, —OH, —OR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl;
each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently and optionally substituted with one or more R;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently and optionally substituted with one or more R;
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently and optionally substituted with one or more R;
or R^{c} and R^{d} are taken together with the atom to which they are attached to form a heterocycloalkyl optionally substituted with one or more R; and
each R is independently deuterium, halogen, —CN, —OH, —OC₁-C₆alkyl, —S(═O)C₁-C₆alkyl, —S(═O)₂C₁-C₆alkyl, —S(═O)₂NH₂, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═O)OC₁-C₆alkyl, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
or two R on the same atom are taken together to form an oxo.

2. The compound of claim 1, or a pharmaceutically acceptable salt, solvate, or stereoisomer thereof, wherein Ring A is a 7- to 8-membered cycloalkyl.

3. The compound of claim 1, or a pharmaceutically acceptable salt, solvate, or stereoisomer thereof, wherein Ring A is a 7-membered cycloalkyl.

4. The compound of claim 1, or a pharmaceutically acceptable salt, solvate, or stereoisomer thereof, wherein Ring A is a 7- to 8-membered heterocycloalkyl comprising 1 to 4 heteroatoms selected from O, S, and N.

5. The compound of claim 1, or a pharmaceutically acceptable salt, solvate, or stereoisomer thereof, wherein Ring A is a 7-membered heterocycloalkyl comprising 1 to 4 heteroatoms selected from O, S, and N.

6. The compound of any one of claims 1-5, or a pharmaceutically acceptable salt, solvate, or stereoisomer thereof, wherein X is N.

7. The compound of any one of claims 1-5, or a pharmaceutically acceptable salt, solvate, or stereoisomer thereof, wherein X is CR^{X}.

8. The compound of any one of claims 1-5 or 7, or a pharmaceutically acceptable salt, solvate, or stereoisomer thereof, wherein R^{X} is halogen.

9. The compound of any one of claims 1-8, or a pharmaceutically acceptable salt, solvate, or stereoisomer thereof, wherein Y is N.

10. The compound of any one of claims 1-8, or a pharmaceutically acceptable salt, solvate, or stereoisomer thereof, wherein Y is CR^{Y}.

11. The compound of any one of claims 1-8 or 10, or a pharmaceutically acceptable salt, solvate, or stereoisomer thereof, wherein R^{Y} is —OH.

12. The compound of any one of claims 1-11, or a pharmaceutically acceptable salt, solvate, or stereoisomer thereof, wherein Z is N.

13. The compound of any one of claims 1-11, or a pharmaceutically acceptable salt, solvate, or stereoisomer thereof, wherein Z is CR^{Z}.

14. The compound of any one of claims 1-11 or 13, or a pharmaceutically acceptable salt, solvate, or stereoisomer thereof, wherein R^{Z} is hydrogen.

15. The compound of any one of claims 1-14, or a pharmaceutically acceptable salt, solvate, or stereoisomer thereof, wherein W is N.

16. The compound of any one of claims 1-15, or a pharmaceutically acceptable salt, solvate, or stereoisomer thereof, wherein each R¹ is independently deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently and optionally substituted with one or more R^{1a}. In some embodiments of a compound of Formula (I), each R¹ is independently deuterium, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, cycloalkyl, or heterocycloalkyl; wherein each alkyl, cycloalkyl, and heterocycloalkyl is independently and optionally substituted with one or more R^{1a}.

17. The compound of any one of claims 1-16, or a pharmaceutically acceptable salt, solvate, or stereoisomer thereof, wherein each R¹ is independently —OH, —OR^{a}, —NR^{c}R^{d}, C₁-C₆alkyl, C₁-C₆haloalkyl, C₁-C₆aminoalkyl, or heterocycloalkyl; wherein each alkyl, and heterocycloalkyl is independently and optionally substituted with one or more R^{1a}.

18. The compound of any one of claims 1-17, or a pharmaceutically acceptable salt, solvate, or stereoisomer thereof, wherein each R¹ is independently —OH, —NR^{c}R^{d}, C₁-C₆alkyl, C₁-C₆aminoalkyl, or heterocycloalkyl; wherein each alkyl, and heterocycloalkyl is independently and optionally substituted with one or more R^{1a}.

19. The compound of any one of claims 1-18, or a pharmaceutically acceptable salt, solvate, or stereoisomer thereof, wherein each R¹ is independently —NR^{c}R^{d} or C₁-C₆alkyl optionally substituted with one or more R^{1a}.

20. The compound of any one of claims 1-19, or a pharmaceutically acceptable salt, solvate, or stereoisomer thereof, wherein each R¹ is independently —NR^{c}R^{d}.

21. The compound of any one of claims 1-19, or a pharmaceutically acceptable salt, solvate, or stereoisomer thereof, wherein each R¹ is independently C₁-C₆alkyl optionally substituted with one or more R^{1a}.

22. The compound of any one of claims 1-19, or a pharmaceutically acceptable salt, solvate, or stereoisomer thereof, wherein each R¹ is independently C₁-C₆alkyl.

23. The compound of any one of claims 1-22, or a pharmaceutically acceptable salt, solvate, or stereoisomer thereof, wherein n is 0 or 1.

24. The compound of any one of claims 1-22, or a pharmaceutically acceptable salt, solvate, or stereoisomer thereof, wherein n is 1 or 2.

25. The compound of any one of claims 1-22, or a pharmaceutically acceptable salt, solvate, or stereoisomer thereof, wherein n is 1.

26. The compound of claim 1, or a pharmaceutically acceptable salt, solvate, or stereoisomer thereof, wherein the compound is selected from a compound of table 1 or table 2.

27. A pharmaceutical composition comprising a compound of any one of claims 1-26, or a pharmaceutically acceptable salt, solvate, or stereoisomer thereof, and a pharmaceutically acceptable excipient.

28. A method of treating cancer in a subject in need thereof, the method comprising administering to the subject an effective amount of a compound of any one of claims 1-26, or a pharmaceutically acceptable salt, solvate, or stereoisomer thereof.

29. A method of treating cancer in a subject in need thereof, the method comprising administering to the subject a pharmaceutical composition of claim 27.

30. The method of claim 28 or 29, further comprising administering an additional therapeutic agent.

31. The method of claim 30, wherein the additional therapeutic agent is an immunotherapeutic agent.

32. The method of claim 31, wherein the immunotherapeutic agent is an anti-PD-1 antibody, an anti-PD-L1 antibody, or an anti-CTLA-4 antibody.

33. A method of treating type-2 diabetes in a subject in need thereof, the method comprising administering to the subject an effective amount of a compound of any one of claims 1-26, or a pharmaceutically acceptable salt, solvate, or stereoisomer thereof.

34. A method of treating type-2 diabetes in a subject in need thereof, the method comprising administering to the subject a pharmaceutical composition of claim 27.

35. A method of treating and/or controlling obesity in a subject in need thereof, the method comprising administering to the subject an effective amount of a compound of any one of claims 1-26, or a pharmaceutically acceptable salt, solvate, or stereoisomer thereof.

36. A method of treating and/or controlling obesity in a subject in need thereof, the method comprising administering to the subject a pharmaceutical composition of claim 27.

37. A method of treating a metabolic disease m a subject in need thereof, the method comprising administering to the subject an effective amount of a compound of any one of claims 1-26, or a pharmaceutically acceptable salt, solvate, or stereoisomer thereof.

38. A method of treating a metabolic disease in a subject in need thereof, the method comprising administering to the subject a pharmaceutical composition of claim 27.
